SEQUENCE LISTING (6 sequences)

SEQ ID NO: 1 - DNA, 26 bp, Artificial Sequence
  XBP1 intron
cagcactcagactacgtgcacctctg

SEQ ID NO: 2 - DNA, 62 bp, Artificial Sequence
  XBP1 intron structure sequence
ccgggtctgctgagtccgcagcactcagactacgtgcacctctgcagcaggtgcaggcccag

SEQ ID NO: 3 - DNA, 201 bp, Artificial Sequence
  XBP1 partial coding sequence, including XBP1 intron
actcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatgg

SEQ ID NO: 4 - DNA, 2231 bp, Artificial Sequence
  RGECO-mNeonGreen calcium detection construct
  misc_feature (1)..(1251): RGECO calcium sensor
  misc_feature (1252)..(1305): T2A protein coding sequence
  misc_feature (1324)..(1524): Partial sequence of XBP1 protein, including intron and intron structure sequences
  misc_feature (1433)..(1494): XBP1 intron structure sequence
  misc_feature (1451)..(1476): XBP1 intron sequence
  misc_feature (1521)..(2231): mNeonGreen
  misc_feature (1525)..(1527): Stop codon before intron is spliced out
atggtcgactcatcacgtcgtaagtggaataaggcaggtcacgcagtcagagctataggtcggctgagctcacccgtggtttccgagcggatgtaccccgaggacggcgccctgaagagcgagatcaagaaggggctgaggctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtgcgaacgcgccgagggccgccactccaccggcggcatggacgagctgtacaagggaggtacaggcgggagtctggtgagcaagggcgaggaggataacagggccatcgtcaaggagttcatgcgcttcaagttgcacatggagggctccgtgaacggccacgagttcgagatcgagggcgagggcgagggccgcccctacgaggcctttcagaccgctaagctgaaggtgaccaagggtggccccctgcccttcgcatgggacatcctgtcccctcagttgatgtacggctccaaggcctacattaagcacccagccgacatccccgactacttcaagctgtccttccccgagggcttcaggtgggagcgcgtgatgaacttcgaggacggcggcattattcacgttagccaggacacctccctgcaggacggcgtattcatctacaaggtgaagctgcgcggcaccaacttcccccccgacggccccgtaatgcagaagaagaccatgggctgggaggctacgcgtgaccaactgactgaagagcagatcgcagaatttaaagaggctttctccctatttgacaaggacggggatgggacgatgacaaccaaggagctggggacggtgatgcggtctctggggcagaaccccacagaagcagagctgcaggacatgatcaatgaagtagatgccgacggtgacggcacattcgacttccctgagttcctgacgatgatggcaagaaaaatgaatgacacagacagtgaagaggaaattagagaagcgttccgcgtgtttgataaggacggcaatggctacatcggcgcagcagagcttcgccacgtgatgacagaccttggagagaagataacagatgaggaggttgatgaaatgatcagggtagcagacatcgatggggatggtcaggtaaactatgaagagtttgtacaaatgatgacagcgaaggagggcagaggaagtcttctaacatgcggtgacgtggaggagaatcccgggcctctagcgtttaccatggttactcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtaa

SEQ ID NO: 5 - DNA, 797 bp, Artificial Sequence
  Single color cell stress biosensor construct
  misc_feature (1)..(273): circularly permuted mNeonGreen exon
  misc_feature (274)..(335): XBP1 intron structure sequence
  Intron (292)..(317): XBP1 intron sequence
  misc_feature (336)..(797): circularly permuted mNeonGreen exon
atggtttcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagggcggtaccccgggtctgctgagtccgcagcactcagactacgtgcacctctgcagcaggtgcaggcccagggagggagcatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtag

SEQ ID NO: 6 - DNA, 1814 bp, Artificial Sequence
  Multiplex fluorescent stress sensor construct
  misc_feature (4)..(24): SV40 nuclear localization signal
  misc_feature (25)..(813): Red fluorescent protein
  misc_feature (814)..(867): T2A sequence
  misc_feature (886)..(1086): Partial sequence of XBP1 protein, including intron and intron structure sequences
  misc_feature (995)..(1056): XBP1 intron structure sequence
  misc_feature (1083)..(1790): mNeonGreen
  misc_feature (1087)..(1089): Stop codon before intron is spliced out
  misc_feature (1791)..(1811): SV40 nuclear localization signal
atgcctaagaagaagaggaaggtcatggtgtcgaagggagaagaactgattaaggaaaacatgcggatgaaagtggtgatggaagggtcggtgaatggacaccaattcaagtgcaccggagagggagagggcaacccatacatgggtactcagaccatgcgcatcaaggtcgttgaaggaggacctctgccttttgcgttcgacatccttgctacctcgttcatgtacgggtcccgcacctttatcaagtacccgaagggaatcccggatttcttcaagcagagcttcccggaaggattcacctgggagagggtgactcggtacgaagatggaggagtgctgactgcaacccaagacacttcgctcgaggacggctgtctggtgtaccatgtccaagtgcggggtgtgaacttcccctcaaatgggccagtgatgcagaaaaagaccctcggatgggaagcgaacaccgagatgatgtacccggcggacggtggcttgcgaggatacactcacatggccttgaagctggacggcggaggtcatctctcatgctcctttgtcactacctaccgcagcaagaaaactgtcggaaacatcaagatgccgggcgtgtactacgtcgatcaccggctcgagagaatcaaagaggccgacaaggaaacgtatgtcgagcagcatgaagtcgcagtggccaggtactgcgaccttccctccaaactgggccacaagctgaattctggcctgagaagccgcgcacaggcttcgaactcagccgtggatgggacggccggcccagggtccactggaagcagagagggcagaggaagtcttctaacatgcggtgacgtggaggagaatcccgggcctctagcgtttaccatggttactcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagcctaagaagaagaggaaggtctaa

(19) United States
(12) Patent Application Publication
(10) Pub. No.: US 2020/0393447 A1
(43) Pub. Date: Dec. 17, 2020

(54) BIOSENSORS FOR MEASURING CELL SIGNALING IN STRESSED AND HEALTHY CELLS

(71) Applicants: Thomas HUGHES, Bozeman, MT (US); Kevin HARLEN, Helena, MT (US); MONTANA MOLECULAR LLC, BOZEMAN, MT (US)

(72) Inventors: THOMAS HUGHES, BOZEMAN, MT (US); KEVIN HARLEN, HELENA, MT (US)

(21) Appl. No.: 16/629,485

(22) PCT Filed: Jul. 6, 2018
(86) PCT No.: PCT/US18/41146

§ 371 Date: Jan. 8, 2020

Related U.S. Application Data
(60) Provisional application No. 62/530,250, filed on Jul. 9, 2017.

(51) Int. Cl. G01N 33/50; C12Q 1/6897

(52) U.S. Cl. CPC G01N 33/5041; G01N 2800/7004; C12Q 1/6897

(57) ABSTRACT

Disclosed herein are nucleic acids comprising reporter constructs for detecting cellular signaling changes in a stressed cell. Also provided are methods for detecting cellular signaling changes in cells undergoing stress, as well as vectors and cells comprising nucleic acids comprising reporter constructs for detecting cellular signaling changes in a stressed cell.

### CROSS-REFERENCE TO RELATED APPLICATIONS

This application claims the benefit of U.S. Provisional Patent Application Ser. No. 62/530,250 filed on Jul. 9, 2017, the entire contents of which are incorporated by reference herein.

### STATEMENT REGARDING FEDERALLY SPONSORED RESEARCH OR DEVELOPMENT

This invention was made with government support under NSF grant IIP-1430879 awarded by the National Science Foundation. The government has certain rights in this invention.

### FIELD

This disclosure relates generally to biological sensors for detecting cell signaling in live cells.

### BACKGROUND

Second messenger signaling is altered in stressed cells. Calcium signaling is shown to be altered in diseased cells, using detection by fluorescent Ca²⁺ dyes (Marambaud et al. 2009). At the biochemical level, altered levels of cyclic adenosine monophosphate (cAMP) have been observed in degenerative neural diseases (Chiu et al. 2015; Sugars et al. 2004; Hebb et al. 2004). In addition, electrophysiology recordings have shown that toxic compounds can affect the resting transmembrane voltage and action potential shapes and durations in excitable cells (Brette et al. 2017; Talbert et al. 2015; Gress et al. 2015).

Genetically encoded, fluorescent biosensors are used to detect changes in intracellular signaling pathways in living cells (see, e.g. U.S. Patent Publication No. 20130298263, U.S. Patent Publication No. 20130344530, U.S. Patent Publication No. 20150037812, and U.S. Patent Publication No. 20170115315, each of which are incorporated by reference). Biosensors may also be used to detect changes in cell membrane voltage (Storace et al. 2016), as well as changes in intracellular second messengers such as cAMP (Tewson et al. 2016), DAG (Tewson et al. 2016), and Ca²⁺ (Akerboom et al. 2013; Zhao et al. 2011). The expression of these sensors has been genetically targeted to specific cellular compartments (Moore et al. 2016), specific cell types (Dana et al. 2014), or whole organs (Ahrens et al. 2013; Tallini et al. 2006).

However, it is currently difficult or impossible to study perturbations in intracellular signaling when the stressed cells are a minor population of the organism, tissue, or culture preparation. For example, in Parkinson's disease, midbrain cultures from animal models contain many cells of different types, only some of which might be undergoing stress at any given moment. This would also be the case for induced pluripotent stem cell-derived neurons from a Parkinson's disease patient. In addition, the level of cellular stress might not be sufficient to allow detection by a standard apoptosis sensor. Consequently, currently available biosensor technology does not allow determination of: 1) which cells are actually stressed; 2) whether the stressed cells are still signaling correctly; 3) whether the stressed cells still respond to certain drugs, and; 4) which new drugs/factors can rescue these cells and enable them to function correctly. Thus, there is clearly a need for improved tools and methods for measuring signaling in cells undergoing stress.

### SUMMARY

The summary of the invention described above is non-limiting and other features and advantages of the invention will be apparent from the following detailed description of the invention, and from the claims.

The instant disclosure provides nucleic acids, cells, vectors, and methods for detecting cells and tissues experiencing endoplasmic reticulum (ER) stress, as well detecting cells and tissues that are experiencing ER stress and changes in their intracellular signaling.

Accordingly, in one aspect, the instant disclosure provides a nucleic acid including from 5′ to 3′: a) a first open reading frame encoding a first reporter protein; b) a linker sequence comprising an intron that is spliced by inositol-requiring enzyme 1 (IRE1) when transcribed to mRNA; and c) a second open reading frame encoding a second reporter protein. The first and second open reading frames are out of frame from each other.

In some embodiments, the intron includes SEQ ID NO: 1. In some embodiments, the intron consists of SEQ ID NO: 1. In some embodiments, the intron includes SEQ ID NO: 2. In some embodiments, the intron consists of SEQ ID NO: 2. In some embodiments, the intron comprises SEQ ID NO: 3. In some embodiments, the intron consists of SEQ ID NO: 3.

In any of the above-mentioned aspects and embodiments, the first reporter protein is a fluorescent or bioluminescent biosensor, the second reporter protein is a fluorescent or bioluminescent biosensor, or both the first and second reporter proteins are fluorescent or bioluminescent biosensors. In some embodiments, the first reporter protein is a fluorescent or bioluminescent biosensor and the second reporter protein is a fluorescent protein. In some embodiments, the first reporter protein is a fluorescent protein and the second reporter protein is a fluorescent or bioluminescent biosensor. In some embodiments, the linker sequence comprises one or more sequence elements selected from the group consisting of an IRES, a 2A peptide, and an alternative translation initiation signal. In some embodiments, the linker sequence encodes a peptide that links the translated first and second reporter proteins when the intron is spliced from the linker sequence mRNA.

In some embodiments, wherein the first reporter protein and the second reporter protein can act as a donor and acceptor pair for resonance energy transfer, wherein the first reporter protein is the donor and the second reporter protein is the acceptor, or the first reporter protein is the acceptor and the second reporter protein is the donor. In some embodiments, the first or second reporter protein may be a fluorescent biosensor, a bioluminescent biosensor, or a fluorescent protein.

In some embodiments, the linker sequence further comprises a sequence encoding a caspase cleavage site. In some embodiments, the first or second fluorescent or bioluminescent biosensor detects changes in the cellular level of a molecule selected from the group consisting of calcium, cyclic adenosine monophosphate (cAMP), cyclic guanylate monophosphate (cGMP), diacylglycerol, adenosine triphosphate (ATP), adenosine diphosphate (ADP), glucose, ribose, sucrose, glutamate, hydrogen peroxide, lactate, magnesium, oxidized nicotinamide adenine dinucleotide (NAD+), non-oxidized nicotinamide adenine dinucleotide (NADH), phosphate, reactive oxygen species, and zinc.

In some embodiments, the first or second fluorescent or bioluminescent biosensor detects changes in the transmembrane voltage of a cell.

In some embodiments of any of the foregoing aspects or embodiments, the first reporter protein is a fluorescent protein, the second reporter protein is a fluorescent protein, or both the first and second reporter proteins are fluorescent proteins.

In some embodiments of any of the foregoing aspects or embodiments, the first open reading frame and the second open reading frame are operatively linked to the same promoter.

In another aspect, the disclosure provides a nucleic acid comprising from 5′ to 3′: a) a first exon of a reporter protein; b) a linker sequence comprising an intron that is spliced by inositol-requiring enzyme 1 (IRE1); and c) a second exon of the reporter protein, wherein the second exon is in a different reading frame from the first exon. In such an aspect, splicing of the intron in mRNA by IRE1 places the second exon in the same reading frame as the first exon.

In some embodiments of the foregoing aspect, the intron comprises SEQ ID NO: 1. In some embodiments, the intron consists of SEQ ID NO: 1. In some embodiments, the intron comprises SEQ ID NO: 2. In some embodiments, the intron consists of SEQ ID NO: 2. In some embodiments, the intron comprises SEQ ID NO: 3. In some embodiments, the intron consists of SEQ ID NO: 3.

In some embodiments, the reporter protein is selected from the group consisting of a fluorescent biosensor, a bioluminescent biosensor, and a fluorescent protein.

In some embodiments, the reporter protein is a fluorescent or bioluminescent biosensor that detects changes in the cellular level of a molecule selected from the group consisting of calcium, cyclic adenosine monophosphate (cAMP), cyclic guanylate monophosphate (cGMP), diacylglycerol, adenosine triphosphate (ATP), adenosine diphosphate (ADP), glucose, ribose, sucrose, glutamate, hydrogen peroxide, lactate, magnesium, oxidized nicotinamide adenine dinucleotide (NAD+), non-oxidized nicotinamide adenine dinucleotide (NADH), phosphate, reactive oxygen species, and zinc.

In some embodiments of the foregoing aspect and embodiments, the fluorescent or bioluminescent biosensor detects changes in the transmembrane voltage of a cell.

In another aspect, the disclosure provides a vector comprising the nucleic acid molecule of any one of the foregoing aspects and embodiments.

In yet another aspect, the disclosure provides a cell including the nucleic acid of any one of the foregoing aspects and embodiments, or the vector comprising the nucleic acid molecule of any one of the foregoing aspects and embodiments.

In some embodiments of the foregoing aspect, the nucleic acid is inserted into the genome of the cell.

In another aspect, the disclosure also provides a kit including the nucleic acid of any one of the foregoing aspects and embodiments, the vector including the nucleic acid of any one of the foregoing aspects and embodiments, or the cell including the nucleic acid of any one of the foregoing aspects and embodiments.

In still another aspect, the disclosure also provides a protein encoded by the nucleic acid of any one of the foregoing aspects and embodiments.

In yet another aspect, the disclosure provides a method for measuring signaling in a cell. The method includes exposing a cell comprising the nucleic acid of any one of the foregoing aspects and embodiments to light having an excitation wavelength of the first reporter protein, and light having an excitation wavelength of the second reporter protein, and measuring the fluorescence from the cell at the emission wavelength of the first reporter protein and at the emission wavelength of the second reporter protein.

In some embodiments of the foregoing method, the method further includes contacting the cell with a molecule or organism selected from the group consisting of: a small molecule; a protein; a bacterium; a virus; a protozoan; a worm; or a fungus.

In some embodiments of the foregoing method, the method further includes exposing the cell to an environmental stressor selected from the group consisting of: a temperature change; a pH change, an osmolarity change; a pressure change; a gravitational force change, and mechanical damage to the cell.

### BRIEF DESCRIPTION OF THE FIGURES

FIG. 1 is a schematic diagram of a two-color multiplex sensor system.

FIG. 2 is a schematic diagram of a second design of another multiplex, two-color sensor system.

FIG. 3A shows photomicrographs of cultured HEK293T cells transfected with a nucleic acid construct of FIG. 1, followed by treatment with carbachol to release calcium stores. Cell stress was induced by treatment with tunicamycin, and stressed cells were identified by expression of mNeonGreen. FIG. 3B shows a graph of the mean fluorescence of an individual unstressed neuron and an individual stressed neuron over time, reflecting the levels of cytosolic calcium detected by the red biosensor.

FIG. 4 shows a graph of the mean fluorescence of stressed and unstressed HEK293T cells transfected with a nucleic acid construct of FIG. 1, followed by treatment with carbachol to release calcium stores. Cell stress was induced by co-expression of a P23H rhodopsin mutant. The top graph shows the calcium response of a stressed HEK293T cell expressing mNeonGreen (indicating ER stress) and the bottom graph shows the calcium response of an unstressed HEK293T cell that did not express mNeonGreen.

FIG. 5 shows a graph of the mean fluorescence of stressed and unstressed HEK293T cells transfected with a nucleic acid construct of FIG. 2 that includes a biosensor for cAMP. Cell stress was induced by co-expression of a P23H rhodopsin mutant. A cAMP response was induced by addition of isoprotenol, indicated by the arrow. The top graph shows the cAMP response of a stressed HEK293T cell expressing mNeonGreen (indicating ER stress) and the bottom graph shows the calcium response of an unstressed HEK293T cell that did not express mNeonGreen.

FIG. 6 shows a schematic diagram of a nucleic acid construct in which an XBP1 intron is inserted within the coding sequence of a circularly permuted mNeonGreen protein. FIG. 6 also shows photomicrographs of HEK293T cells transfected with the nucleic acid construct, both before (top photomicrograph) and after (bottom photomicrograph) being treated with thapsigargin to induce ER stress.

FIG. 7 is a schematic diagram of a nucleic acid that uses FRET to detect both ER stress and apoptosis signaling in a cell.

FIG. 8A shows photograph of an acrylamide gel of RT-PCR amplification products for spliced and unspliced mRNA from cells transfected with a fluorescent biosensor and treated with thapsigargin to induce ER stress. FIG. 8B shows photomicrographs of HEK293T cells transduced with the two-color stress sensor and treated with either 1 μM thapsigargin or DMSO. FIG. 8C shows graphs of the change in fluorescence over time for transfected HEK293T cells treated with either thapsigargin or DMSO. The top graph shows the change in green fluorescence, the middle graph show the change in red fluorescence, and the bottom graph shows the change of the red/green fluorescence ratio.

FIG. 9A shows photomicrographs of iPSC-derived peripheral neurons transduced with a fluorescent biosensor and treated with various concentrations of vincristine to induce ER stress. FIG. 9B is a box plot of the percentage of stressed cells, as indicated by green fluorescence, following treatment with vincristine. FIG. 9C is a dose response curve for vincristine treatment of iPSC-derived peripheral neurons and calculated EC50 value. FIG. 9D is comparison of EC50 values of four different chemotherapeutics calculated by measuring cell stress and compared to IC50 values calculated by measuring neurite outgrowth.

FIG. 10 shows graphs of fluorescence intensity for HEK293T cells transfected with a two-color fluorescent biosensor and either a wild-type (WT) or mutant version of the rhodopsin, SOD, or alpha-synuclein genes. Graphs for green fluorescence, indicating stress levels, red fluorescence, indicating overall protein expression levels, and green/red ratio are shown for each pair of wild type and mutant genes.

### DETAILED DESCRIPTION

The technology described herein provides nucleic acid constructs that link the stress status of a cell with the cell's signaling properties. In particular, the disclosure describes fluorescent sensor systems that can be used to both identify a cell experiencing stress and measure the signaling properties of the stressed cell. Once obtained, the signaling properties of the stressed cell can be compared with the signaling properties with healthy, unstressed cells, allowing a user to determine the effect of stress on cellular signaling properties. As described further below, nucleic acids encoding the fluorescent sensor systems described herein can be introduced into a cell to allow detection of the cell's stress status and its intracellular signaling as detected by the sensor. Such sensor systems comprise first and second reporter proteins encoded by an engineered nucleic acid. The first and second reporter proteins may be a fluorescent protein, a bioluminescent protein, or a fluorescent biosensor, wherein expression of the first or second protein is dependent on the stress status of the cell.

Accordingly, a general embodiment of the instant technology is a nucleic acid molecule having first and second open reading frames encoding first and second reporter proteins, one of the reporter proteins being a fluorescent biosensor, and the second reporter protein being a fluorescent protein or another fluorescent biosensor, wherein a linker sequence between the two open reading frames comprises an intron that interrupts the protein coding sequence. Removal of the intron by splicing is dependent on the stress status of the cell.

### Definitions

Unless defined otherwise, all technical and scientific terms used herein have the same meaning as commonly understood by one of ordinary skill in the art.

As used herein, the term “nucleic acid” refers to a polymer of two or more nucleotides or nucleotide analogues (such as ribonucleic acid having methylene bridge between the 2′-O and 4′-C atoms of the ribose ring) capable of hybridizing to a complementary nucleic acid. As used herein, this term includes, without limitation, DNA, RNA, LNA, and PNA. A nucleic acid may be single-stranded or double-stranded. Where the nucleic acid is single-stranded, a skilled person in the art will appreciate that, the nucleic acid can be in the sense or antisense orientation relative to the direction of transcription of the reporter genes.

As used herein, the term “gene” refers to a nucleic acid sequence that encodes an amino acid sequence. A gene of the invention can include a nucleic acid sequence that is a contiguous coding sequence (e.g., an open reading frame; ORF), as well as nucleic acid sequences that contain exons and introns. In the context of the instant technology, the term gene can, but need not, include regulatory sequences such as, for example, promoter sequences, enhancer sequence, polyadenylation signals, and the like.

Genes of the instant technology can be joined to regulatory sequences, such as promoters, thereby allowing expression of the genes. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters, e.g. beta-actin promoter or EF1α promoter, or from hybrid or chimeric promoters (e.g., CMV promoter fused to the beta-actin promoter). Promoters from the host cell or related species are also useful herein.

As used herein, the term “enhancer” generally refers to a sequence of DNA that functions at no fixed distance from the transcription start site and can be either 5′ or 3′ to the transcription unit. Furthermore, enhancers can be within an intron as well as within the coding sequence itself. Enhancers are usually between 10 and 300 base pairs in length, and function in cis. Enhancers usually function to increase transcription from nearby promoters; in some species (e.g. D. melanogaster), enhancers can function in trans on a corresponding allele on another chromosome. Enhancers can also contain response elements that mediate the regulation of transcription. While many enhancer sequences are known from mammalian genes (globin, elastase, albumin, fetoprotein, and insulin), typically enhancers from a eukaryotic cell virus are used for general expression. Preferred examples are the SV40 enhancer on the late side of the replication origin, the cytomegalovirus early promoter enhancer, the polyoma enhancer on the late side of the replication origin, and adenovirus enhancers.

Genes in constructs of the invention can be operatively linked to the same promoter, or each gene can be independently, operatively linked to a different promoter. As used herein, the term “operatively linked” means that the promoter can direct the expression of a linked sequence, which encodes protein. In one embodiment, a first gene and a second gene are operatively linked to the same promoter. The promoter and/or an enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

In one embodiment, transcription from the promoter results in production of a polycistronic mRNA molecule comprising the first gene sequence and the first and second exon sequences. As used herein, the term “polycistronic mRNA” is a mRNA molecule that carries multiple, independent coding regions that can produce multiple, independent proteins. There are many ways of creating such mRNAs, which include, without limitation, internal ribosomal entry sites (IRES), 2A peptide sequences (Szymczak et al. 2004), or strategically positioned alternative translation start signals. In one embodiment, the nucleic acid molecule comprises an internal ribosomal entry site (IRES) sequence upstream of the first exon. In one embodiment, the nucleic acid molecule comprises a sequence encoding a 2A peptide sequence upstream of the first exon.

As used herein, the term “reporter protein” refers to a protein that is detectable by a user when expressed by a cell in a non-truncated form, and the term “reporter gene” refers to a gene encoding a reporter protein. For example, a reporter protein may be a fluorescent protein that fluoresces when exposed to a certain wavelength of light (e.g. GFP, enhanced GFP). A reporter protein may be fluorescent biosensor that changes its fluorescence properties in response to a particular type of cell signaling. A reporter protein may be an enzyme that catalyzes a reaction with a substrate to produce an observable change in that substrate, such as the luminescence enzyme luciferase which acts on luciferin or other substrates to emit photons, or β-galactosidase which can hydrolyze X-gal (5-bromo-4-chloro-3-indolyl-β-D-galactopyranoside) to form a blue precipitate that can visualized.

As used herein, the term “bioluminescent protein” refers to a protein that catalyzes a reaction with a substrate to emit photons, without needing a light source to excite the protein. Exemplary bioluminescent proteins include, but are not limited to: luciferase (e.g. from fireflies, jellyfish, or dinoflagellates), aequorin (which emits photons when oxidized in the presence of calcium).

As used herein, the term “fluorescent protein” refers to a protein that emits light at some wavelength after excitation by light at another wavelength. Exemplary fluorescent proteins that emit in the green spectrum range include, but are not limited to: green fluorescent protein (GFP); enhanced GFP (EGFP); superfolder GFP; AcGFP1; and ZsGreen1; and mNeonGreen. Exemplary fluorescent proteins that emit light in the blue spectrum range include, but are not limited to: enhanced blue fluorescent protein (EBFP), EBFP2, Azurite, and mKalama1. Exemplary fluorescent proteins that emit light in the cyan spectrum range include, but are not limited to: cyan fluorescent protein (CFP); enhanced CFP (ECFP); Cerulean; mHoneydew; and CyPet. Exemplary fluorescent proteins that emit light in the yellow spectrum range include, but are not limited to: yellow fluorescent protein (YFP); Citrine; Venus; mBanana; ZsYellow1; and Ypet. Exemplary fluorescent proteins that emit in the orange spectrum range include, but are not limited to: mOrange; tdTomato; Exemplary fluorescent proteins that emit light in the red and far-red spectrum range include, but are not limited to: DsRed; DsRed-monomer; DsRed-Express2; mRFP1; mCherry; mStrawberry; mRaspberry; mPlum; E2-Crimson; iRFP670; iRFP682; iRFP702; iRFP720. Far red fluorescent proteins (e.g. iRFP670; iRFP682; iRFP702; iRFP720) can be useful for animal or thick tissue preparations, as the wavelength is able to penetrate these thicker structures. Such far red proteins can be engineered into biosensors (see, e.g. Shcherbakova et al. 2016, fusing miRFP703 to IκBα to detect NF-κB activation). Exemplary listings of fluorescent proteins and their characteristics may be found in Day and Davidson. 2009, and in Rodriguez et al. 2017, each of which is incorporated herein by reference.

Fluorescent proteins may include chimeric combinations of fluorescent proteins that transfer and receive energy through fluorescent resonance energy transfer (FRET) when exposed to a particular wavelength of light. In some embodiments, an acceptor in a FRET pair may emit light at a certain wavelength after accepting energy from a donor molecule exposed to another wavelength of light. Exemplary chimeric FRET pairs, include, but are not limited to ECFP-EYFP; mTurquoise2-SeYFP; EGFP-mCherry; and Clover-mRuby. In some embodiments, the acceptor molecule of chimeric fluorescent molecule may quench the light emission of a donor molecule exposed to its preferred wavelength of light. Quenching between different portions of chimeric fluorescent proteins may occur using a photoactivatable acceptor. For example, a chimeric fluorescent protein may include a photoactivatable GFP that can then quench photoemission by CFP. Examples of FRET proteins are discussed in Hildebrandt et al., Sensors (Base1). 2016 September; 16(9): 1488, incorporated herein by reference.

Fluorescent proteins may also include chimeric combinations of bioluminescent proteins and fluorescent proteins that transfer and receive energy through bioluminescent resonance energy transfer (BRET). In some embodiments, an acceptor in a BRET pair (e.g. GFP) may emit light at a certain wavelength after accepting energy from photons emitted by a bioluminescent protein (e.g. luciferase). In such an embodiment, the bioluminescent protein alone, before ER-stress induced splicing, would produce light of one particular wavelength. After the mRNA is spliced to remove the intron, a new, closely linked fluorescent protein would be translated that would accept the energy emitted by the bioluminescent protein and in turn emit light of different wavelength. For example, luciferase fused to a fluorescent protein and catalyzing luciferin or an analogue would produce a red-shifted light. In this case the luciferase emission alone would be one marker, and the red-shifted emission from the acceptor protein, if it is there, would be the second signal (see, e.g., Xu et al. 1999).

As used herein, the term “fluorescent biosensor” (also referred to as a cell signaling sensor protein) refers to a recombinant, fluorescent fusion protein that changes its fluorescence properties in response to a particular type of cell signaling. For example, in the case of excitable cells, a fluorescent biosensor may change fluorescence in response to changes in transmembrane voltage, such as FlaSh (a voltage-gated potassium channel fused to a fluorescent protein), ArcLight (a voltage-sensitive phosphatase fused to a mutated pHluorin), and microbial rhodopsin-based proteins that are either inherently fluorescent or can be paired with a fused fluorescent protein to utilize FRET fluorescence or quenching (e.g. Mermaid, using fluorescent proteins from Coral) (see, e.g. Storace et al. 2016, incorporated herein by reference). Alternatively, the biosensor may change fluorescence in response to changes in the level of a cell signaling molecule such as, for example, calcium (e.g. Cameleon, a fusion of calmodulin, calmodulin-binding peptide, and GFP), chloride (e.g. Clomeleon, a fusion of a chloride-sensing yellow fluorescent protein and cyan fluorescent protein), pH (e.g. pHluorin), cAMP (see, e.g. U.S. Patent Application No. 20160274109A1 incorporated herein by reference), cGMP (see, e.g., Nikolaev et al. 2006, incorporated herein by reference), or diacylglycerol (DAG) (see, e.g., U.S. Pat. No. 9,547,017 incorporated herein by reference). FLIP biosensors utilize binding proteins from bacteria (e.g. glutamate/aspartate binding protein, glucose binding protein, sucrose binding protein) fused to two GFPs (see, e.g. Okumuto et al. 2005; Bermejo et al. 2010; Lager et al. 2006). HyPer (a circular permutant of YFP) and roGFP (with substituted cysteines) can be used for detection of reactive oxygen species (see e.g. Bilan et al. 2013). REX-YFP or Peredox (a fusion of fluorescent protein and the T-Rex sensor from Thermus aquaticus) may be used to detect the redox state of nicotinamide adenine dinucleotide (NAD+/NADH) (see, e.g. Bilan et al. 2014; Hung et al. 2011). Zinc can be detected using a fusion of a fluorescent protein and a His4 protein sensor (see, e.g. Dittmer et al. 2009). Phosphate detection may be accomplished using bacterial phosphate-binding protein (PiBP) to eCFP and eYFP (see, Gu et al. 2006.). Perceval is a fusion of bacterial regulatory protein GlnK1 and cpmVenus (eYFP) for detecting the ATP/ADP ratio in live cells (see, e.g., Berg et al., 2009). FLIPW is a fusion of tryptophan-activated repressor protein (TrpR) and eCFP and cpmVenus (eYFP) (see e.g. Kaper et al. 2007). Intracellular lactate may be detected with a fusion of bacterial L1d receptor and Venus (eYFP) (see, e.g., San Martin et al. 2013.). MagFRET is a fusion of human centrin 3 (HsCen3) to Cerulean and Citrine that detects magnesium (Lindenburg et al. 2013). Exemplary signaling molecules that may be detected by a fluorescent biosensor include, but are not limited to, calcium chloride, cAMP, cGMP, diacylglycerol, ATP, ADP, glucose, ribose, sucrose, glutamate, hydrogen peroxide, lactate, magnesium, NAD+, NADH, phosphate, reactive oxygen species, and zinc.

As used herein, the term “cellular stress” refers to a wide variety of molecular changes that occur in cells in response to environmental stressors such as, for example, temperature changes, changes in pH, changes in osmolarity, changes in pressure, changes in gravitational force, mechanical damage to the cell, infection, and the like. Environmental stressors may also include cell contact with molecules and organisms, including, but not limited to small molecules (a molecule of molecular weight less than 900 daltons, e.g. chemotherapeutic compounds), proteins (e.g. diphtheria toxin), bacteria, viruses, protozoa, worms, and fungi. Cells respond to stressors, at least in part, by modulation of cellular molecules, one class of which are stress response proteins. Many of these stress response proteins affect the transcription of certain genes through either direct interaction with genetic elements (e.g., promoters), or through modulation of other molecules (e.g., proteins) that eventually interact with genetic elements. Thus, the “stress status” of a cell can refer to the relative level and/or activity of one or more stress response proteins. Accordingly, the stress status of a cell can be determined by measuring the level or activity of one or more stress response proteins. Such measurement can be direct (e.g., determination of protein level) or indirect (e.g., assays using reporter constructs affected by one or more stress response proteins).

Examples of stress response proteins include, but are not limited to, x-box binding protein 1 (XBP1), and inositol requiring protein 1 (IRE1), both of which are activated when hydrophobic patches are exposed in unfolded proteins in the lumen of the endoplasmic reticulum (described in further detail below). The presence of unfolded proteins activates the RNase activity of IRE1, which splices immature, cytosolic mRNA for XBP1, generating an active transcription factor that alters gene expression (Yoshida et al. 2001). Thus, in one embodiment, a stressed cell is a cell in which the RNAse activity of the IRE1 protein has been activated. In one embodiment, a stressed cell is a cell in which the XBP1 intron is being spliced. This is classically referred to as the unfolded protein response (UPR); however, a wide variety of stimuli can cause this splicing, so the broader term of stressed cells is used here. For example, reactive oxygen species are known to produce the UPR (Santos et al. 2009), as well as metabolic stressors (Pilkis et al. 1992; Belmadani et al. 2017). Mis-folded proteins often found in neurodegenerative diseases trigger the UPR (Halliday et al. 2014). Substances and drugs that are toxic to cells often trigger the UPR (Foufelle et al. 2016).

As used herein, the term “linker sequence” or “linker nucleic acid” refers to a nucleotide sequence that is located between two other nucleotide sequences. In some embodiments, a linker nucleotide sequence may encode an intron, an IRES, a cleavage site of a protease, and/or a ribosomal skipping peptide. For example, if two proteins are translated with a linker nucleotide sequence encoding an amino acid sequence of a protease cleavage site (e.g. 3C or “PreScission”; enterokinase (EKT); Factor Xa (Fxa); Tobacco etch virus (TEV); or thrombin) from a multicistronic mRNA, specific proteases that are concomitantly expressed by the cell will cleave the peptide at the cleavage site. In another example, the nucleotide sequence encoding peptide 2A (such as T2A, P2A, E2A, or F2A) may be used, which causes ribosomal skipping at the end of the 2A sequence. This leaves no peptide bond between the 2A peptide and any peptide translated after the 2A peptide, and allows multiple separate proteins to be translated from a single transcript.

As used herein, the term “exon” refers to a nucleic acid sequence that encodes a peptide or protein sequence. In some embodiments, an exon encodes part of a protein sequence. In some embodiments, an exon encodes an entire protein sequence.

As used herein, the term “intron” refers to a nucleic acid sequence that interrupts other coding sequences, such as exons. Any intron can be used in constructs of the technology described herein, as long as activation of the cell stress response causes the intron to be spliced out of pre-mRNA.

As used herein, the term “splicing” refers to the removal of introns from a pre-mRNA sequence.

As used herein, the term “pre-mRNA” refers to messenger RNA (mRNA) transcribed from a nucleic acid molecule, which has not yet undergone splicing and thus contains at least one intron. As used herein, the term “mature mRNA” refers to pre-mRNA that has completed the splicing process and is ready to undergo translation to produce an encoded protein. In one embodiment, the intron is recognized by the IRE1 protein. In one embodiment, the intron is a cytoplasmic XBP1 intron present in the XBP1 mRNA. In one embodiment, the intron comprises a sequence at least 50%, at least 60%, at least 70%, at least 80%, at least 85%, at least 90%, at least 95%, at least 97% or at least 99% identical to SEQ ID NO: 1 or SEQ ID NO:2. In one embodiment, the intron comprises SEQ ID NO: 1 or SEQ ID NO: 2. In one embodiment, the intron is SEQ ID NO: 1 or SEQ ID NO: 2.

As used herein, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like, in connection with the recitation of claim elements, or use of a “negative” limitation.

As used herein, the term “about” in quantitative terms refers to plus or minus 10% of the value it modifies (rounded up to the nearest whole number if the value is not subdividable, such as a number of molecules or nucleotides). For example, “about 100 mg” would encompass 90 mg to 110 mg, inclusive; “about 2500 mg” would encompass 2250 mg to 2750 mg. When applied to a percentage, the term “about” refers to plus or minus 10% relative to that percentage. For example, “about 20%” would encompass 15-20% and “about 80%” would encompass 75-85%, inclusive. Moreover, where “about” is used herein in conjunction with a quantitative term it is understood that in addition to the value plus or minus 10%, the exact value of the quantitative term is also contemplated and described. For example, the term “about 23%” expressly contemplates, describes, and includes exactly 23%.

### Unfolded Protein Response (UPR)

The unfolded protein response (UPR) is a cellular stress mechanism for managing unfolded protein accumulation in the lumen of the endoplasmic reticulum (ER), which can occur when protein synthesis is unregulated due to protein mutation or other stress conditions. Binding immunoglobulin protein (BiP), also known as glucose regulated protein 78 (GRP78), acts as an initial sensor of unfolded proteins in the ER and initiates the UPR pathway.

During times of normal ER function, BiP is associated with the luminal domain of three different ER membrane proteins: inositol-requiring enzyme 1 (IRE1), activating transcription factor 6 (ATF6), and PKR-like ER kinase (PERK). If BiP interacts with a hydrophobic, unfolded patch of protein in the lumen of the ER, it dissociates from IRE1, ATF6, and PERK, allowing these luminal ER proteins to act as part of the unfolded protein response. Following dissociation from BiP, IRE1 oligomerizes and autophosphorylates, activating RNase activity that splices many mRNAs, including immature, cytosolic mRNA for the transcription factor X-box binding protein 1 (XBP1). Spliced XBP1 generates an active transcription factor that alters gene expression (Yoshida et al. 2001).

This cytosolic splicing of XBP1 mRNA has been used to generate reporter systems in which XBP1 splicing produces a functional, fluorescent protein (Iwawaki et al. 2003; U.S. Patent Publication Number US2012/0202751A1, incorporated herein by reference), and there are now a variety of PCR-based splicing assays or reporters of XBP1 transcriptional activity (Rong et al. 2015).

ER stress and the UPR can be induced by a number of drugs, including anticancer drugs (Foufelle et al. 2016; Pluquet et al. 2015) and mutations associated with different diseases, especially neurodegenerative diseases (Grootjans et al. 2016; Scheper et al. 2015; Halliday et al. 2014; Wang et al. 2012) and cancer (Cubillos-Ruiz et al. 2017).

### Unfolded Protein Response (UPR)

One embodiment of the technology described herein is a nucleic acid comprising a gene comprising a first exon and a second exon separated by an intron, wherein the intron is chosen such that activation of the cellular stress response results in splicing of the intron and joining of the first and second exons to form an open reading frame (ORF) that encodes a fluorescent biosensor. In such an embodiment, the level of fluorescence from the fluorescent biosensor changes in response to the cellular level of a cell signaling molecule such as, for example, a cell signaling molecule selected from the group consisting of Ca²⁺, cAMP, cGMP, diacylglycerol, ATP, ADP, glucose, glutamate, hydrogen peroxide, lactate, magnesium, NAD+, NADH, phosphate, reactive oxygen species, ribose, sucrose, and zinc. In certain embodiments, the level of fluorescence from the fluorescent biosensor changes in response to the transmembrane voltage of the cell.

In certain embodiments, nucleic acid constructs encoding a multiplex fluorescent biosensor would express a genetically encoded, fluorescent sensor for diacylglycerol or Ca²⁺ in all of the cells, and a different fluorescent protein in only the cells undergoing a stress response that causes splicing of the XBP1 intron. Such nucleic acid constructs would allow comparison of diacylglycerol or Ca²⁺ signaling in healthy and stressed cells (See Example 1). In stressed cells the diacylglycerol or Ca²⁺ levels could be different due to effects on either the sources of diacylglycerol or Ca²⁺ or the clearance of these second messengers. Such fluorescent biosensors could also be used to identify whether stressed cells have a different pharmacological profile such that drugs that typically cause diacylglycerol or Ca²⁺ signaling in healthy cells no longer do in stressed cells. The fluorescent biosensors could also be used to determine if there are drugs that would act preferentially on diacylglycerol or Ca²⁺ signaling in stressed cells rather than healthy ones.

In certain embodiments, the two-colored multiplex sensor would express a genetically encoded, green fluorescent sensor for cAMP in all of the cells, and red fluorescent protein in just the ones undergoing an unfolded protein response that causes splicing the of XBP1 intron (SEQ ID NO:1). This would allow comparison of cAMP signaling in healthy and stressed cells (Example 2). In stressed cells the cAMP levels could be different due to effects on either the sources of cAMP, the adenylyl cyclases, or the clearance of cAMP by phosphodiesterases. The sensor could also be used to identify whether stressed cells have a different pharmacological profile such that drugs that typically cause cAMP signaling in healthy cells no longer do in stressed cells. The sensor could also be used to determine if there are drugs that would act preferentially on cAMP signaling in stressed cells rather than healthy ones.

In certain embodiments, a single transcript would carry encode of the necessary components, including both a fluorescent biosensor, a different colored fluorescent protein, and the XBP1 intron (SEQ ID NO:1). In a different embodiment separate vectors would comprise two different genes that produce two different transcripts, one of which includes the XBP1 intron and one color of fluorescent protein and a different transcript that carries a different colored fluorescent sensor for cell signaling.

One embodiment of the invention is a nucleic acid molecule comprising a nucleic acid sequence at least 80%, at least 85%, at least 90%, at least 95%, at least 97% or at least 99% identical to a nucleic acid sequence disclosed herein. One embodiment of the invention is a nucleic acid molecule comprising any of the nucleic acid sequences disclosed herein.

One embodiment of the invention is a nucleic acid comprising a nucleic acid sequence at least 80%, at least 85%, at least 90%, at least 95%, at least 97% or at least 99% identical to SEQ ID NO:3 or SEQ ID NO:4. One embodiment of the invention is a nucleic acid molecule comprising SEQ ID NO:3 or SEQ ID NO:4.

One embodiment of the invention is a protein encoded by a nucleic acid of the invention.

The present disclosure also discloses nucleic acid vectors useful for practicing methods of the invention. In certain embodiments, a nucleic acid vector of the invention can be a plasmid, a cosmid, a viral vector, and the like, that comprises a nucleic acid molecule of the invention. The nucleic acid constructs described herein may be introduced into a cell using transient transfection techniques (e.g. using a plasmid introduced by lipids or electroporation), or it may be stably integrated into a cellular genome, such as by viral delivery (e.g. using a lentivirus or baculovirus vector). The multifunctional DBS reporter constructs may also be integrated into a specific genomic region of interest, using site-directed recombinase technology (e.g. Cre-Lox or FLP-FRT) or transposon-based technology (e.g. Sleeping Beauty transposon/SB100X).

Nucleic acid molecules of the invention may be inserted into the genomes of transgenic animals or model organisms, used to create stable cell lines, or transiently expressed via transfection or viral transduction. In such constructs, the nucleic acid molecule may be inserted into the host genome or remain episomal. Methods to generate stable lines or animals or to transiently express the sensors are well known in the art and readily adaptable for use with the compositions and methods described herein. Nucleic acid sequence of the invention may also located in the genome of a cell in a transgenic animal and tissue, including, but not limited to, C. elegans, drosophila, mice, rats, marmosets, organoids, and embryonic stem cells derived from any vertebrate.

Methods of making cells of the invention are known and the method of transformation and choice of expression vector will depend on the host system selected. Transformation and transfection methods are described, e.g., in F. Ausubel et al., Current Protocols in Molecular Biology, John Wiley & Sons, New York, N.Y., (1998), and, as described above, expression vectors may be chosen from examples known in the art. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein. Examples of viral vectors useful for practicing the present invention include, but are not limited to, Adenovirus, Adeno-associated virus, Lentivirus, Baculovirus modified for mammalian expression (BacMam), herpes virus, Vaccinia virus, Polio virus, Sindbis, and other RNA viruses, including these viruses with the HIV backbone. Non-viral based vectors, can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs.

As described above, sensor systems of the invention are useful for measuring cell signaling (e.g., Ca²⁺, cAMP, cGMP, diacylglycerol, ATP, ADP, glucose, glutamate, hydrogen peroxide, lactate, magnesium, NAD+, NADH, phosphate, reactive oxygen species, ribose, sucrose, zinc, etc.) in cells that are undergoing stress. Thus, one embodiment of the invention is a method of measuring cell signaling in a cell undergoing a stress response, comprising exposing a cell comprising a nucleic acid molecule of the invention to light having the excitation wavelength of the first fluorescent protein and the second fluorescent protein, and measuring the fluorescence from the cell at the emission wavelength of the first fluorescent protein and the second fluorescent protein.

One embodiment of the present invention is a kit for practicing methods of the present invention. Kits can include a nucleic acid molecule, a nucleic acid vector, or a cell of the present invention as well components for making such nucleic acid molecules, nucleic acid vectors, or cells. As such, kits can include, for example, primers, nucleic acid molecules, expression vectors, DNA constructs of the present invention, cells, buffers, reagents, and directions for using any of said components. It should be appreciated that a kit may comprise more than one container comprising any of the aforementioned, or related, components. For example, certain parts of the kit may require refrigeration, whereas other parts can be stored at room temperature. Thus, as used herein, a kit comprises components sold in separate containers by one or more entity, with the intention that the components contained therein be used together.

### Two-Color Multiplex Reporter Construct

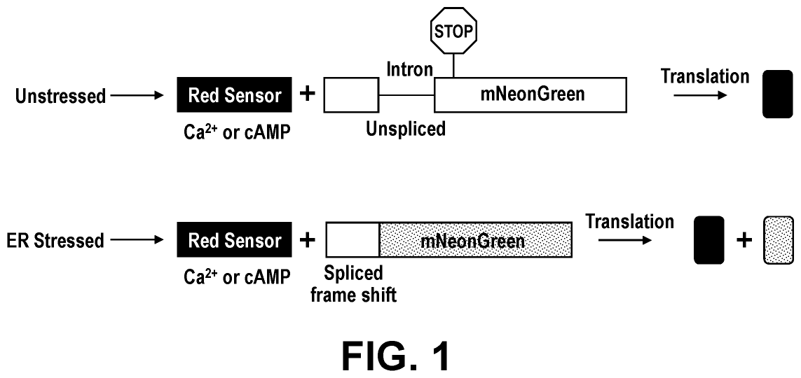

An exemplary two-color multiplex reporter nucleic acid construct is shown in the schematic diagram of FIG. 1 (from 5′ to 3′). The nucleic acid encodes an open reading frame for a fluorescent biosensor that fluoresces in the red spectrum while in the presence of intracellular Ca²⁺ or cyclic adenosine monophosphate (cAMP). The red biosensor could be any biosensor that detects a second messenger molecule, protein, metabolite, or other small molecule. Instead of a biosensor, the encoded protein may be a fluorescent protein (e.g., see Example 5 discussed below). The emission spectrum of the fluorescent biosensor or fluorescent protein may be any emission spectrum that is distinguishable from the second fluorescent biosensor or fluorescent protein encoded in the construct. The sequence encoding the red biosensor protein is followed by an intron sequence and a stop codon. A second open reading frame for the fluorescent protein mNeonGreen follows the intron sequence, but is out of frame from the red biosensor (see top portion of diagram in FIG. 1). While mNeonGreen is the fluorescent protein exemplified in FIG. 1, any fluorescent protein or fluorescent biosensor with a different emission spectrum from the first fluorescent biosensor (or fluorescent protein) may be used.

While a cell is unstressed, i.e. is not mobilizing or expressing proteins in the UPR (e.g. XBP1 or IRE1), the red biosensor is expressed constitutively, and the mNeonGreen protein is not translated because its coding sequence is out of frame. In the cells undergoing ER stress, IRE1 is released from the BiP, dimerizing and acquiring RNase activity. IRE1 recognizes the intron in transcribed RNA and splices it out (see bottom portion of diagram in FIG. 1). Splicing the intron from the transcribed mRNA brings the coding sequence for mNeonGreen into the same frame as the red biosensor, resulting in the expression the mNeonGreen protein in addition to the red biosensor. This is a modular design in which there is a constitutively expressed fluorescent biosensor, and fluorescent protein with a distinct signal that is only expressed in stressed cells.

In some embodiments, the intron is the XBP1 intron CAGCACTCAGACTACGTGCACCTCTG (SEQ ID NO: 1). The XBP1 intron is the sequence spliced out of the mRNA by IRE1 during the unfolded protein response. In some embodiments, the intron includes the XBP1 intron structure sequence SEQ ID NO: 2 CCGGGTCTGCTGAGTCCGCAGCACTCAGACTACGTGCACCTCTGCAGCAGGTGCA GGCCCAG (SEQ ID NO: 2). The XBP intron structure sequence includes the XBP1 intron that is spliced out of the pre-mRNA, and includes sequence that is recognized by IRE1 during the splicing event. In some embodiments, the intron includes a portion of the XBP1 protein coding sequence and the XBP1 intron structure sequence (SEQ ID NO: 3).

A nucleic acid construct of FIG. 1 is exemplified by the RGECO-mNeonGreen calcium detection construct of SEQ ID NO: 4. From 5′ to 3′, a sequence encoding red calcium biosensor (RGECO: red fluorescent protein fluorescent genetically encoded Ca²⁺ indicator for optical imaging) is followed by a sequence for T2A peptide to separate the RGECO protein from the downstream mNeonGreen. The T2A peptide is then followed by a partial XBP1 protein sequence that includes the XBP1 intron. An open reading frame for mNeonGreen is also encoded, out of frame from the RGECO/XBP1 sequence.

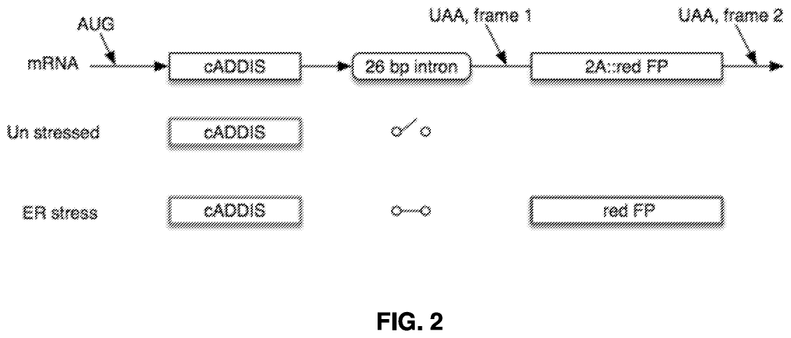

FIG. 2 shows a second design of a two-color multiplex reporter construct, from 5′ to 3′ (see top portion of FIG. 2 labeled “mRNA”). An open reading frame encoding a green fluorescent biosensor that fluoresces in the presence of cAMP (labeled cADDis; see e.g. US Patent Publication number 20160274109A1, incorporated by reference herein) is followed by an intron 26 base pairs long (e.g., the XBP1 intron and/or intron structure sequence). The intron sequence is then followed by a sequence that is out of frame and encodes a 2A peptide and red fluorescent protein (RFP). When a cell comprising this nucleic acid construct is in an unstressed state, only the green cAMP biosensor (cADDis) is expressed. However, if the cell is experiencing ER stress and is undergoing the UPR, mobilized IRE1 recognizes and splices the intron from the mRNA, leading to a frameshift and translation of the red fluorescent protein. The 2A peptide then self-cleaves, releasing the red fluorescent peptide from the green biosensor.

### EXAMPLES

The following examples are meant to be illustrative and should not be construed as further limiting. The contents of the figures and all references, patents, and published patent applications cited throughout this application are expressly incorporated herein by reference.

### Example 1

### Multiplex Ca²⁺ Biosensor Construct to Measure Ca²⁺ Response in Cells Exposed to Stressor Molecule

In order to analyze the calcium response of cells exposed to an ER-stress inducing molecule, HEK293T cells transfected with the nucleic acid construct described in FIG. 1 were exposed to thapsigargin to induce ER stress. Thapsigargin is an inhibitor of the sarcoplasmic/endoplasmic reticulum Ca²⁺ ATPase that inhibits the autophagic process and induces ER stress. The calcium signaling properties of the thapsigargin-treated cells was measured as described below.

HEK293T cells were transfected with SEQ ID NO: 4 as follows. 27,000 cells per well in 100 μL of EMEM media were plated onto Greiner 96-well plates coated with poly-D lysine. The cells were incubated for 24 hours at 37° C. and 5% CO₂. 100 ng of plasmid DNA expressing SEQ ID NO: 4 and 10 ng of plasmid expressing human muscarinic receptor 1 were then transfected using FUGENE as follows. 100 ng of DNA expressing SEQ ID NO: 4 and 10 ng of DNA expressing human muscarinic receptor 1 were combined with 0.4 μL of FUGENE and brought to a final volume of 50 μL using OPTIMEM serum free media and incubated for 20 mins at 25° C. then added to the cells. After 16 hours the cells were treated with a final concentration of 1 μM thapsigargin for 5 hours to induce ER stress. The cells were then washed 5× with 100 μL DPBS and 150 μL of DPBS was added to the wells. The cells were imaged using a Zeiss Axiovert fluorescence microscope using standard Green Fluorescent Protein (GFP) and Red Fluorescent Protein (RFP) excitation and emission filters. Images were obtained in both the red and green channels every 2 seconds for 100 seconds. A final concentration of 50 μM carbachol was added to the wells to stimulate Gq signaling after 20 seconds, releasing intracellular Ca²⁺ stores for detection by the biosensor. The images were analyzed as follows. Cells were identified as either stressed or unstressed based on their green fluorescence intensity. The Ca²⁺ signaling response upon Gq stimulation was monitored in the red fluorescence channel.

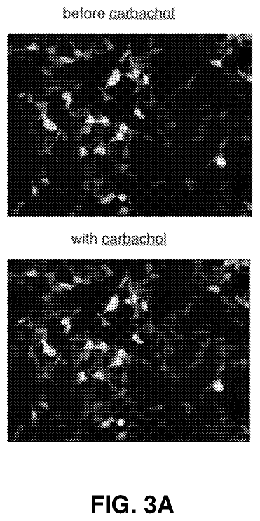

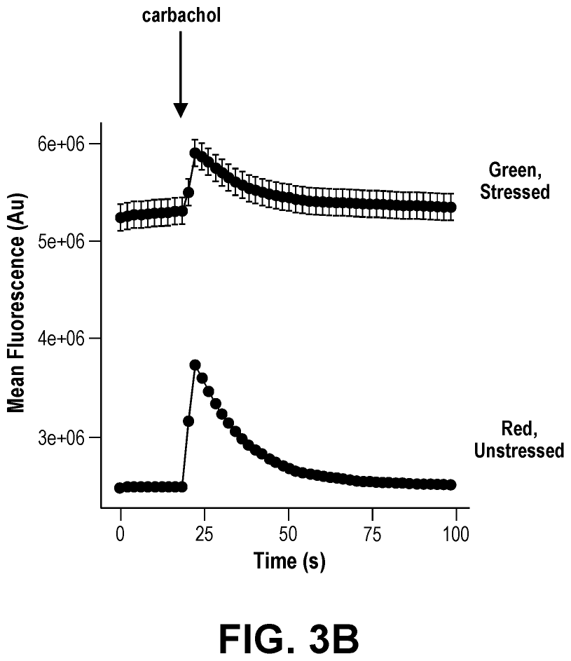

In the ER stressed cells, splicing of the XBP1 intron introduces a frameshift in the coding region such that the mNeonGreen protein is expressed, marking the cells that experience ER stress. FIG. 3A shows photomicrographs of HEK293T cell cultures treated with thapsigargin (subjected to ER stress) before carbachol treatment (top photomicrograph) and after carbachol treatment (bottom photomicrograph). The cells express both the red fluorescent biosensor (constitutively), and the mNeonGreen protein due to the ER stress induced by the thapsigargin treatment. In the top photomicrograph, red fluorescence of the biosensor is at a low level, due to the low levels of intracellular calcium. The bottom photomicrograph of FIG. 3A shows a visible increase in red fluorescence due to intracellular Ca²⁺ release by Gq receptor stimulation by carbachol.

FIG. 3B shows a graph of mean fluorescence intensity over time before and after carbachol treatment, in a neuron expressing mNeonGreen and experiencing ER stress (top graph), and in a neuron that is unstressed (not expressing mNeonGreen; bottom graph). The time of carbachol treatment of the cells is noted by the arrow. FIG. 3B clearly shows that ER-stressed neurons expressing the mNeonGreen protein have a higher resting level of cytosolic Ca²⁺ and that these neurons, when compared to surrounding healthy cells, have a blunted, smaller amplitude response to release of Ca²⁺ stores.

### Example 2

### Multiplex Ca²⁺ Biosensor Construct to Measure Ca²⁺ Response in Cells Stressed with Mutant Protein

In order to analyze the calcium response of cells expressing a mutant protein, HEK293T cells were transfected with both the nucleic acid construct described in FIG. 1 and a mutant rhodopsin protein (P23H). The P23H mutation in rhodopsin causes retinitis pigmentosa (Rivolta et al. 2002), an inherited form of blindness that develops gradually over the first decade of life.

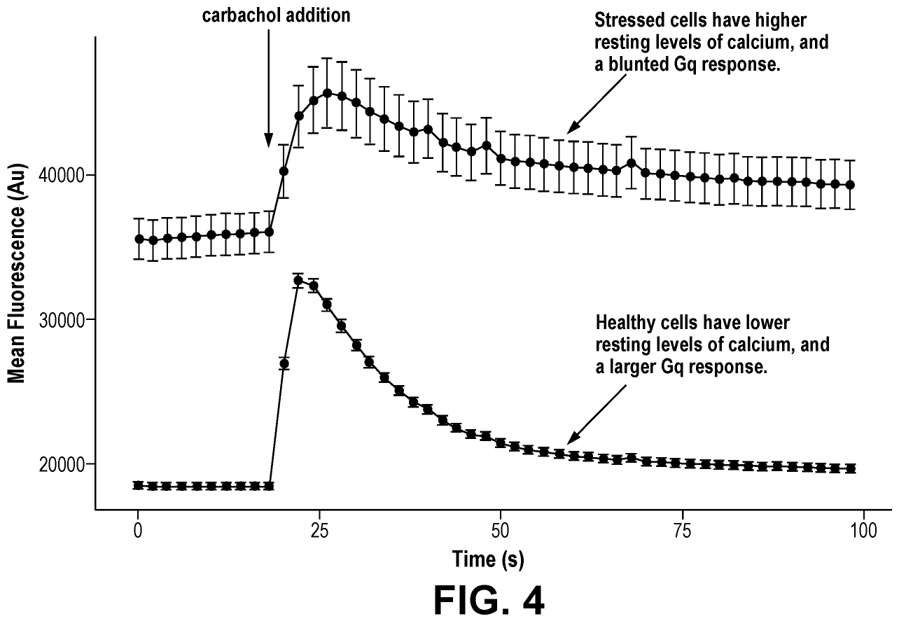

The experiment in FIG. 4 was conducted the same as in FIG. 3 with the following modifications. 100 ng of a plasmid expressing the P23H rhodopsin mutation was co-transfected with the plasmid expressing SEQ ID NO: 4 and 10 ng of human muscarinic receptor 1. After 24 hours the cells were treated with 50 μM carbachol as described in Example 1 above, and the Ca²⁺ signaling was monitored in the red fluorescence channel for cells identified as stressed or unstressed based on their green fluorescence intensity.

FIG. 4 shows a graph comparing changes in mean fluorescence intensity of the biosensor before and after carbachol treatment over time, in an individual ER stressed HEK293T cell expressing mNeonGreen (top graph) and an unstressed HEK293 cell expressing only the red fluorescent Ca²⁺ biosensor (bottom graph). The time of carbachol treatment of the cells is noted by the arrow in the graph of FIG. 4. Cells expressing the P23H rhodopsin mutant show very different Ca²⁺ handling. Similar to the tunicamycin treatment in Example 2 above, the resting cytosolic levels of Ca²⁺ are higher in the stressed cells compared to the unstressed cells, and the release of intracellular Ca²⁺ stores following carbachol treated is blunted in the stressed cells compared to the unstressed cells.

### Example 3

### Multiplex cAMP Biosensor Construct in HEK293 Cultures

In order to analyze the cAMP response of cells exposed to a ER-stress inducing molecule, HEK293T cells were transfected with 100 ng of a red fluorescent cAMP biosensor (cADDis; Montana Molecular, Bozeman, Mont., USA) and 100 ng of the rhodopsin P23H mutant plasmid described in Example 2 above. After 24 hours of incubation, the green and red fluorescence were monitored on a BioTek synergy MX plate reader with the fluorescence intensity read every 30 seconds. Isoproterenol was added after 5 minutes to stimulate Gs signaling and increase intracellular levels of cAMP, and the cells were monitored for 15 minutes after isoproterenol addition.

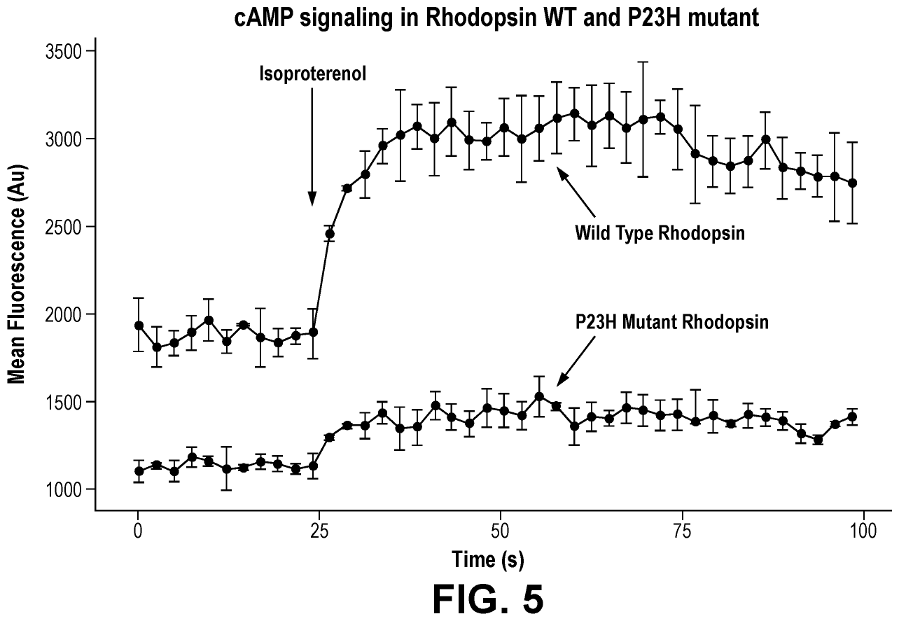

FIG. 5 shows graph comparing changes in mean fluorescence intensity of the cAMP biosensor (cADDis) in transfected cells before and after isoproterenol treatment, in an individual ER stressed HEK293 cell expressing mNeonGreen (top graph) and an unstressed HEK293 cell expressing only the red cADDis cAMP biosensor (bottom graph).

Expression of the rhodopsin mutant P23H not only produces changes in Ca²⁺ response to carbachol as discussed in Example 2, but also produces changes in cAMP signaling. In this case, the ER stressed cells expressing P23H rhodopsin have lower levels of cytosolic cAMP, and the response of the cells to a drug that produces large increases in cAMP in healthy cells (turquoise) is much smaller in the stressed ones (red). This is consistent with the observation that cAMP dependent processes are reduced in cells stressed in neurodegenerative diseases (Carlyle et al. 2014; Bibb et al. 2000).

### Example 4

### Single Color Cell Stress Biosensor Construct in HEK293 Cultures

In order to determine if a fluorescent protein can be used with the intron splicing behavior of IRE1, a nucleic acid construct was created in which the XBP1 intron is inserted within a circularly permuted mNeonGreen protein coding sequence. Circularly permuted fluorescent proteins have been incorporated into biosensor proteins to fluoresce in response to the presence of an intracellular molecule, such as DAG (see, e.g. U.S. Pat. No. 9,547,017B2, incorporated herein by reference) and cAMP (see, e.g. US Patent Publication number 20160274109A1, incorporated herein by reference).

The XBP1 region inserted into the circularly permuted mNeonGreen coding sequence includes both the spliced intron and a coding sequence for several amino acids from XBP1. Following mRNA splicing of the XBP1 intron, a coding portion of the XBP1 sequence remains. The spliced mRNA encodes several amino acids, and there are several regions in the fluorescent protein that can tolerate the insertion of additional amino acids while maintaining the ability of the protein to fluoresce.

HEK293T cells were transfected with SEQ ID NO: 5 as follows. 27,000 cells per well in 100 μL of EMEM media were plated onto Greiner 96-well plates coated with poly-D lysine. The cells were incubated for 24 hours at 37° C. and 5% CO₂. The cells were transfected with 100 ng of plasmid DNA expressing SEQ ID NO: 5 using FUGENE as follows. 100 ng of DNA was combined with 0.4 μL of FUGENE and 47.6 μL of OPTIMEM serum-free media and incubated for 20 mins at 25° C. then added to the cells. After 16 hours the cells were treated with a final concentration of 1 μM thapsigargin (to induce ER stress) or a DMSO control (vehicle control) for 5 hours. The green fluorescence was then imaged using a Zeiss Axiovert fluorescence microscope and standard GFP excitation and emission filters.

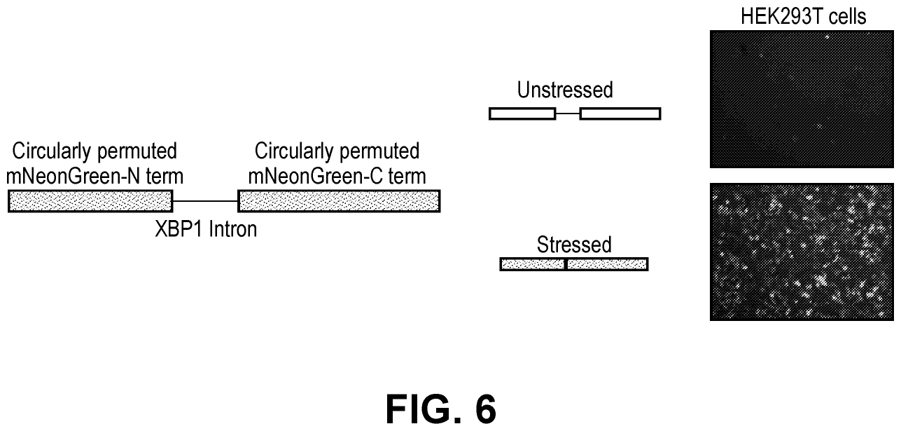

FIG. 6 shows a schematic diagram of a nucleic acid construct in which an XBP1 intron is inserted within the coding sequence of a circularly permuted mNeonGreen protein. The photomicrograph next to the “unstressed” schematic diagram (the intron is not spliced out of the mRNA) shows that the cells are not fluorescing, and thus do not produce functional mNeonGreen protein. The photomicrograph next to the “stressed” schematic diagram (the intron is spliced out of the mRNA), shows that the transfected cells are expressing functional mNeonGreen protein, indicating that they are experiencing an ER stress condition.

### Example 5

### Multiplex FRET Biosensor Construct to Detect Cell Stress, Intracellular Signaling, and Apoptosis

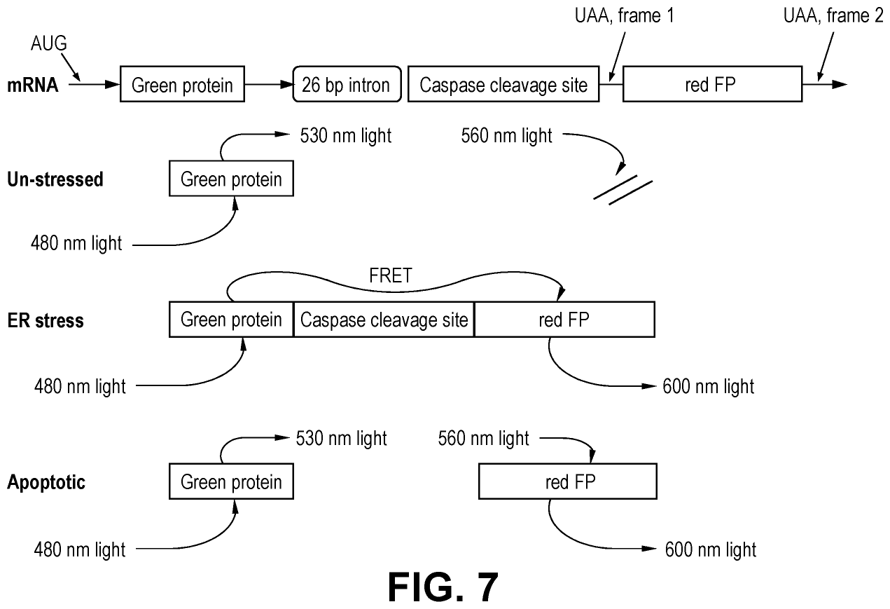

FIG. 7 illustrates an example of how a multicolored, multifunctional Fluorescence Resonance Energy Transfer (FRET) system could be created. The diagram labeled “mRNA” at the top of the page details the construction of the construct, from 5′ on the left end to 3′ on the right end. Constructs may be made using any nucleic acid. A sequence encoding a fluorescent green protein (such as mNeonGreen) is encoded in a first open reading frame, followed by an intron that includes a termination codon, such as the XBP1 intron. This is followed by a sequence encoding a caspase cleavage site, and then a red fluorescent protein. Both the caspase cleavage site and the red fluorescent protein coding sequence are in a second open reading frame that is out of frame from the first open reading frame. Any two fluorescent proteins or fluorescent biosensors that have different fluorescence emission spectra and that can act as FRET donors and acceptors may be used.

As shown in FIG. 7 in the schematic labeled “Unstressed,” unstressed, healthy cells transfected with this nucleic acid construct would only produce a green fluorescent protein and there would be no red fluorescence, as the intron and stop codon prevents further translation from the mRNA. If the cells are undergoing a ER stress response, the intron is spliced out by mobilized IRE1, allowing the caspase cleavage amino acid sequence and red fluorescent protein to be expressed as a fusion protein with the green fluorescent protein. Thus, stressed cells would produce both green and red proteins that readily exchange energy through FRET, because they would be joined as a single protein, positioning the green and red proteins close enough to one another for FRET to occur (see FIG. 7, schematic labeled “ER Stress”). In the example illustrated in FIG. 7, ER stressed transfected cells excited with 480 nm light leads to emission of light at a wavelength of 600 nm, indicating that the cell is undergoing ER stress.

If a cell is committed to die via apoptosis, caspases expressed as part of the apoptotic signaling pathway would then recognize and cleave the caspase cleavage site in the translated FRET fusion protein, eliminating the FRET response of the fusion protein (see FIG. 7, schematic labeled “Apoptotic”). In the example illustrated in FIG. 7, cell excitation with 480 nm light results in emission of 530 nm light from the separated green protein, a light emission that is not seen in cells with an intact FRET fusion protein. This system can provide insights into whether the cells were healthy, stressed but surviving, or marked for certain death through apoptosis.

### Example 6

### Signal Reversibility of Multiplex Fluorescent Stress Sensor Construct

A second nucleic acid construct (SEQ ID NO: 6) was constructed and tested. This nucleic acid construct is a red fluorescent protein (RFP) with a nuclear localization sequence, placed upstream of the XBP1 intron sequence. A T2A peptide encoding sequence is placed after the RFP sequence. This is followed by a partial sequence from the endogenous XBP1 gene that encodes a region of the protein important for regulating its stability. This partial XBP1 sequence is fused to the mNeonGreen coding sequence. Upon stress activation the XBP1 intron sequence is removed, this removes a stop codon, shifts the reading frame, and allows for translation of the XBP1-mNeonGreen fusion construct. This construct was transfected into HEK293T cells, which were then treated with 1 μM thapsigargin or DMSO vehicle control to induce ER stress. Isolated mRNA from the treated cells was then analyzed for intron splicing using RT-PCR (see van Schadewijk et al., 2012).

HEK293T cells were transduced with 25 μL of the nucleic acid construct of SEQ ID NO: 6. The construct was transduced into 48,000 HEK293T cell in FluoroBrite media and the cells were incubated at 37° C. and 5% CO₂ for 16 hours. A final concentration of 1 μM thapsigargin was added to the cells. For RT-PCR analysis a total of 2 μg of total RNA was isolated form 6 wells of cells grown on 96-well Greiner plates coated in poly-D-lysine. RNA was isolated using the Zymo Quick RNA microprep kit according to the manufacturer's instructions. mRNA was converted to cDNA by reverse transcription using M-MLV reverse transcriptase from Promega and an oligo dT primer according to the manufacturer's instructions. RNA was isolated at 30 minutes, 2.5 hours, 6 hours, and 24 hours after treatment with thapsigargin. After cDNA conversion primers spanning the XBP1 intron sequence specific to the biosensor were used to determine the level of XBP1 splicing at each time point. PCR products were then analyzed on a 2% agarose gel. Cells were also imaged at the same time points used to collect RNA for RT-PCR analysis. All images were obtained using a Zeiss Axiovert fluorescence microscope with standard RFP and GFP excitation and emission filter sets. The red and green images were overlaid in Fiji image analysis software. To continuously monitor cells treated with thapsigargin over 24 hours HEK293T cells were transduced and treated with either DMSO or thapsigargin as described above. The media was supplemented with 25 mM HEPES and the cells were incubated in the BioTek Synergy MX plate reader at 37° C. for 24 hours. Green fluorescence was monitored using excitation and emission wavelengths of 485 nm and 528 nm respectively, while red fluorescence was monitored using 558 nm/603 nm excitation and emission wavelengths. All wavelengths had a bandpass of 20 nm. The percentage of change in green, red, and green/red ratio fluorescence was then calculated.

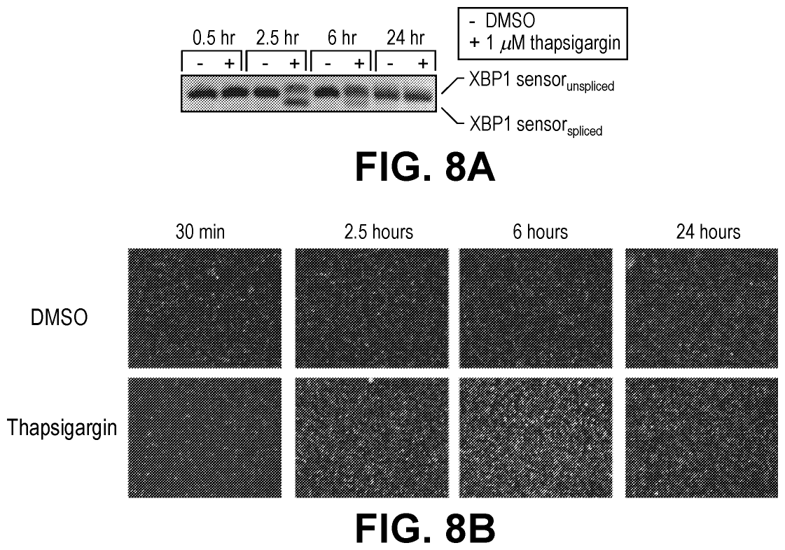

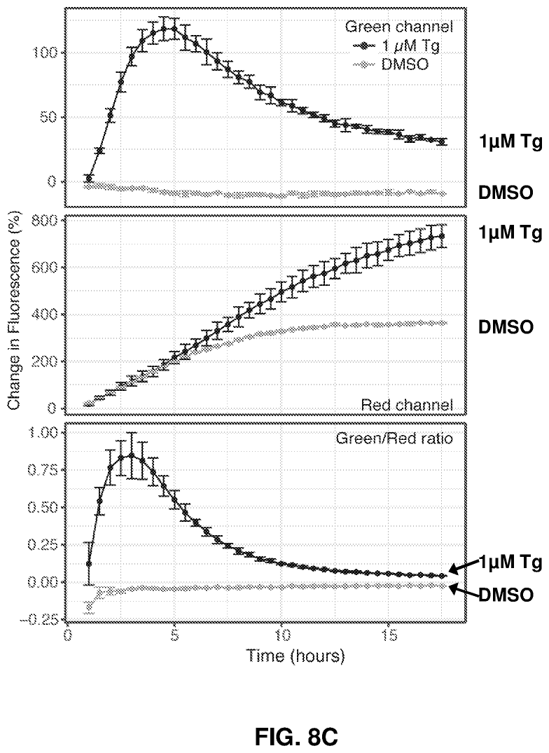

FIG. 8A shows an acrylamide gel of RT-PCR amplification product from mRNA isolated from the transduced HEK293T cells treated with either DMSO or thapsigargin. Treated cells were analyzed at 30 minutes. 2.5 hours, 6 hours, and 24 hours after exposure to thapsigargin. RT-PCR using primers spanning the intron show the presence and absence of the spliced form of the sensor. Importantly, the spliced version of the sensor (the lower band) appears only in the thapsigargin treated cells where ER stress has been activated, peaking in intensity at 2.5 hours post-treatment, and disappears after 24 hours, when the cells have recovered from stress. The splicing of the sensor mimics the response of the endogenous XBP1 gene in cells treated with thapsigargin, recovering from stress within 24 hours as indicated by the lack of the spliced XBP1 isoform.

FIG. 8B shows photomicrographs of HEK293T cells transduced with the two-color stress sensor and treated with either 1 μM thapsigargin or DMSO. Images are overlays of both the red and green fluorescence channels. These changes are quantified for individual cells, as shown by the bar graphs of FIG. 8C, FIG. 8C shows graphs of the change in fluorescence over time for transfected HEK293 cells treated with either thapsigargin or DMSO. The top graph shows the change in green fluorescence, the middle graph show the change in red fluorescence, and the bottom graph shows the change of the red/green fluorescence ratio. Similar to the splicing dynamics of the sensor observed using the RT-PCR splice detection technique, the green stress induced fluorescence appears rapidly, then disappears by 24 hours.

The experiments above show that the addition of this sequence instills the reversible nature of the sensor. The reversibility of the signal by the biosensor is important for two at least two reasons. First, the biosensor can detect both the onset and recovery from ER stress, allowing the sensor to be used for both the detection of stress-inducing compounds or mutations, as well as compounds that reverse ER-mediated cell stress. Second, the biosensor allows kinetic monitoring of stress onset and recovery. When screening drug compounds or testing new compounds for ER-stress activation the kinetics of ER stress activation and cellular recovery are important. Accordingly, fluorescent biosensors disclosed herein can be used to determine both in a single assay.

Without being bound by theory, the reversibility of the XBP1-mNeonGreen signal likely comes from the fact that the XBP1 spliced protein (XBP1s) is an unstable version of XBP1. Stability for the endogenous version of XBP1 is regulated first by binding of the unspliced version of XBP1 to the spliced version, which causes the spliced version to relocate to the cytoplasm from the nucleus and be degraded (see, e.g., Yoshida et al. 2006). The second mechanism that stabilizes the spliced version of XBP1 is the binding of the UBC9 protein to the leucine zipper domain (residues 93-133) of XBP1. In this instance UBC9 binds to the leucine zipper domain and stabilizes the XBP1s protein form by preventing its degradation (see, e.g., Uemura et al. 2013).

In the case of the sensor described in Example 6 (RFP-XBP1-mNeonGreen), residues 123-189 of the unspliced form of XBP1 protein are used in the construct. This version of XBP1 in the construct is unstable and rapidly degraded (see Yoshida et al. 2006). Under the endogenous mechanism the XBP1 leucine zipper domain stabilizes XBP1s after its splicing and protects it from being bound by the unspliced form, exclusion from the nucleus, and degradation. As the XBP1 sequence in the construct of SEQ ID NO: 6 does not contain this leucine zipper domain, it is not stabilized by UBC9 and thus still subject to binding by the unspliced XBP1 isoform, removal from the nucleus, and degradation by the proteasome. Importantly, as SEQ ID NO: 6 does not encode the full length XBP1 protein, it is only the endogenous XBP1 protein of the transfected cell that is regulating degradation of the XBP1-mNeonGreen protein. This means that the XBP1-mNeonGreen protein is responding to the endogenous state of ER stress. Endogenous XBP1 from the cell's ER stress response continually clears the XBP1-mNeonGreen fusion protein from the nucleus for degradation in the cytosol. As ER stress decreases and endogenous XBP1 is no longer mobilized, the XBP1-mNeonGreen protein is also not created by mRNA splicing. This results in a decrease in the cellular XBP1-mNeonGreen protein to undetectable levels when measuring fluorescence intensity, as any remaining XBP1-mNeonGreen is degraded.

### Example 7

### Determining Percentage of ER-Stressed Cells in a Population

The two-color biosensor described in Example 5 above (RFP-GFP stress sensor; SEQ ID NO: 6) may be used to calculate the percent of cells undergoing ER stress, and may be used as measurement technique to detect side effects induced by chemotherapeutics. In order to determine the percentage of cells in population experiencing ER-stress following exposure to a ER-stress-inducing compound, iPSC-derived peripheral neurons were transduced with the two-color stress sensor described in Example 5 above. The cells were treated with the chemotherapeutic vincristine at concentrations of 0.0001 μM, 0.01 μM, and 0.1 μM. Measurement of the red fluorescence was used to identify transduced cells and measurement of green fluorescence identifies stressed cells. Stressed and unstressed cells were counted to quantify the percentage of stressed cells in the population.

Peri.4U peripheral neurons from Ncardia were plated onto 96-well plates according to the manufacturer's instructions. Cells were incubated for 2 days at 37° C. and 5% CO₂ and the media was changed every 24 hours, using Ncardia neuro supplement media. Cells were then transduced with 25 μL of the two-color cell stress sensor (SEQ ID NO:6) and incubated for 16 hours. Varying concentrations of the chemotherapeutic vincristine or DMSO were added to the cells in 8 replicates for each condition. After 24 hours of incubation with the drug, a final concentration of 1 mM crystal Ponceau 6R was added to the cells and they were imaged as described above in Example 5. Image analysis in Cellprofiler was used to determine the percentage of stressed cells in each condition. Those cells that expressed both the constitutively expressed red nuclear marker and the green, stress induced, nuclear fluorescence were considered stressed, while those only expressing the red marker were considered unstressed. The percentage of stressed in each condition was also used to determine EC50 values for vincristine.

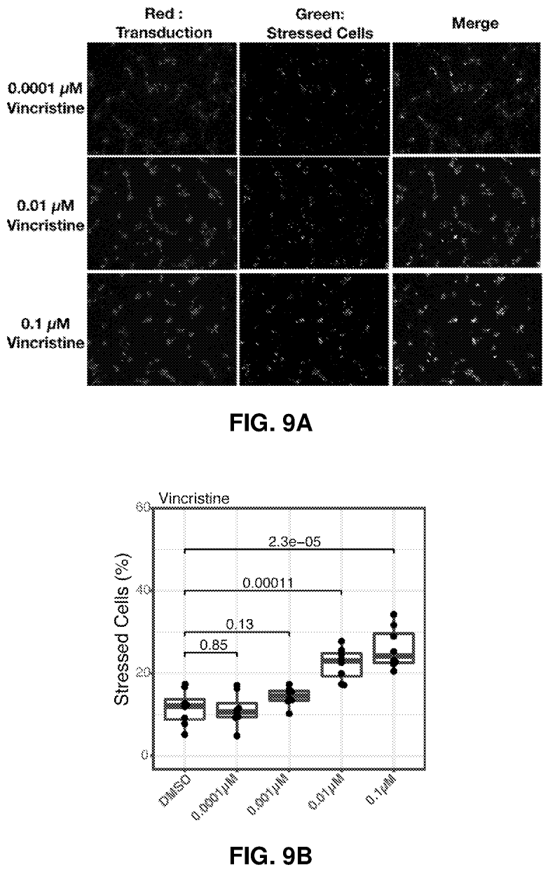

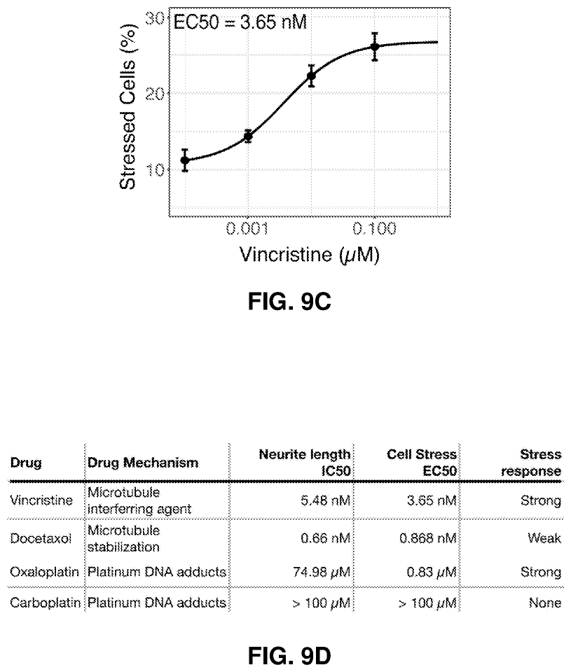

FIG. 9A shows photomicrographs of the vincristine-treated cells to detect the red fluorescent protein and the green fluorescent protein (left column is the red protein, middle column is the green protein, and the right column is a merged composite of both the red and green photomicrographs).

FIG. 9B is a box plot quantifying the percentage of cells within a population that are stressed, with p-values relative to the control treatment also shown. In this example, treatments of 0.01 μM and 0.1 μM vincristine resulted in statistically significant percentages of stressed cells compared to control treatment (p<0.00011 and p<2.3e−05, respectively).

The data from FIG. 9B was used to calculate an effective concentration (EC50) for the iPSC-derived peripheral neurons receiving the two doses of vincristine treatment. FIG. 9C shows the calculated dose response curve for vincristine treatment of iPSC-derived peripheral neurons and the calculated. EC50 value.

This method of calculating an EC50 for stress-induction was compared with another technique that measures inhibition of neurite growth by cells in response to compound exposures. Peripheral neurons derived from iPSCs were treated with the chemotherapeutic compounds vincristine (a microtubule-interfering agent), docetaxel (a microtubule stabilizing agent), oxaloplatin, and carboplatin (platinum DNA adducts), and neurite growth inhibition was measured using the methods described in Rana et al., 2017, to calculate an IC50. Images were captured and analysis was performed after 24 h of drug treatment and drug treated cultures were compared to time-matched, untreated controls. Image analysis was performed using the Neuronal Profiling version 4 bioapplication, and all fields were evaluated for each well. DAPI was used to identify and count individual nuclei in channel 1. βIIItubulin was used in channel 2 to identify cell bodies and neurites by intensity. Cell bodies were not used for neurite detection if they contained>1 nucleus. The critical well level output parameters reported were Neuron Count per Valid Field (provides relative cell counts for cytotoxicity measurement), and Mean Neurite Total Length Ch2. Concentration-response plots were expressed as percent of time-matched controls (equivalent endpoints in untreated cultures) plotted as log of concentration vs. normalized response. IC50 values for each compound were generated using a non-linear regression model of the form Y=100/(1+10\hat{ }((Log IC50.X)* Hillslope))), constrained between 0 and 100% of the time matched controls.

FIG. 9D shows a table comparing, for each compound, the calculated IC50s using neurite growth inhibition and the calculated EC50s using the two-color biosensor technique described above in this example.

The EC50 values determined using the cell stress sensor are very similar to the inhibitory concentrations (IC50) values determined using the neurite outgrowth assay (data not shown). For the chemotherapeutic compound oxaloplatin, the stress sensor is two orders of magnitude more sensitive that neurite outgrowth.

### Example 8

### Detecting Genetically Induced ER-Stress

The biosensors described herein can be used to simultaneously monitor changes in both ER-mediated stress and overall protein expression. As stress-inducing mutations or stress caused by chemical compounds can often change protein expression within the cell having indicators of both stress and protein expression is important for determining actual causes of cellular stress.

To analyze ER-mediated stress and protein expression, HEK293T cells were transfected with the RFP-GFP stress sensor described above in Example 6, along with plasmids encoding either the wild type or a mutant version of a gene selected from rhodopsin, alpha-synuclein, and superoxide dismutase 1 (SOD1). The green and red fluorescence of the transfected cells was measured, and the ratio of green to red fluorescence was calculated.

HEK293T cells were transfected with SEQ ID NO: 6 and either wild type or mutant versions of rhodopsin, SOD or alpha-synuclein genes as follows. 27,000 cells per well in 100 μL of FluoroBrite media were plated onto Greiner 96-well plates coated with poly-D lysine. The cells were incubated for 24 hours at 37° C. and 5% CO₂. The cells were then transfected with 100 ng of each plasmid using LIPOFECTAMINE 2000 according to the manufacturer's instructions. The cells were incubated for 24 hours as described above. The green and red fluorescence for triplicate wells of cells expression the two-color stress sensor and either wild type or mutant versions of the rhodopsin, SOD1, or alpha-synuclein genes was then acquired using a BioTek Synergy MX plate reader. Green fluorescence was monitored using excitation and emission wavelengths of 485 nm and 528 nm respectively, while red fluorescence was monitored using 558/603 excitation and emission wavelengths. All wavelengths had a bandpass of 20 nm. The mean green, red and green/rad ratio fluorescence and standard deviation were then plotted for each condition.

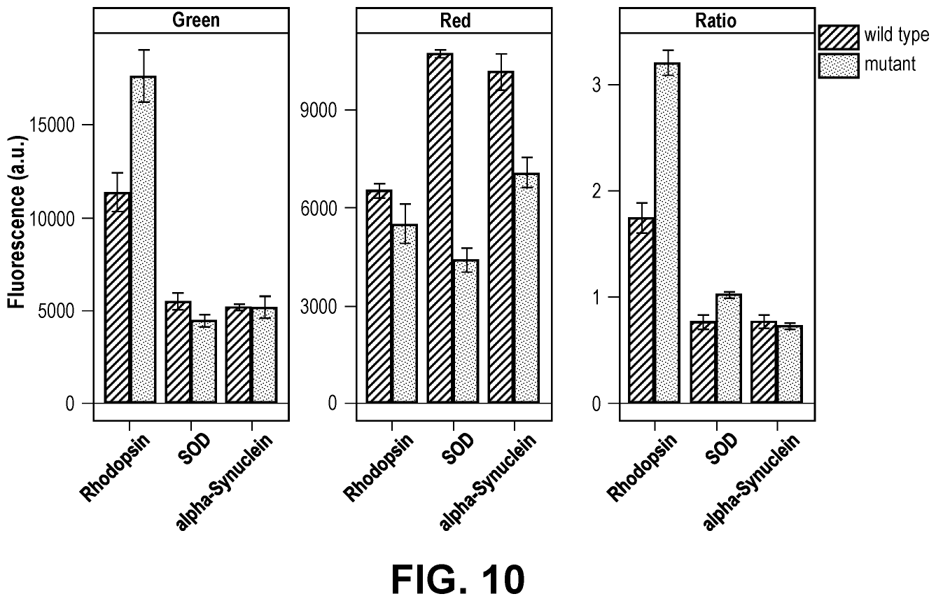

FIG. 10 shows bar graphs comparing the fluorescence intensity between wild type and mutant proteins in the transfected HK293T cells. Analysis of the green fluorescence quantifies changes in ER-mediated cell stress between the mutant and wild-type (WT) versions of each gene (left bar graph of FIG. 10). Only the mutant rhodopsin protein shows a significant stress-induced fluorescence, indicated by the increase in green fluorescence over wild-type rhodopsin protein.

The red fluorescence is used to monitor overall changes in protein expression caused by expression of the mutant genes (middle bar graph of FIG. 10), as the red fluorescent protein is constitutively expressed regardless of the ER-stress state of the cell. The mutant versions of SOD1 and alpha-synuclein show a significant decrease in protein expression compared to their wild-type counterparts.

The green/red fluorescence ratio measures the cumulative effects of changes in ER-stress and protein expression in the transfected cells. The mutant rhodopsin protein showed the greatest difference in cumulative effects.

The foregoing description is intended to illustrate but not to limit the scope of the disclosure, which is defined by the scope of the appended claims. Other embodiments are within the scope of the following claims.

### REFERENCES

Ahrens et al. 2013. “Whole-Brain Functional Imaging at Cellular Resolution Using Light-Sheet Microscopy.” Nat. Methods 10(5):413-20.
Akerboom et al. 2013. “Genetically Encoded Calcium Indicators for Multi-Color Neural Activity Imaging and Combination with Optogenetics.” Front Mol Neurosci. 6 (January): 1-29.
Belmadani et al. 2017. “The Unraveling Truth About IRE1 and MicroRNAs in Diabetes.” Diabetes 66(1): 23-24.
Berg et al., 2009. “A genetically encoded fluorescent reporter of ATP:ADP ratio.” Nat Methods. 6(2): 161-166.
Bermejo et al. 2010. “Dynamic analysis of cytosolic glucose and ATP levels in yeast using optical sensors.” Biochem J. 432(2):399-406;
Bibb et al. 2000. “Severe Deficiencies in Dopamine Signaling in Presymptomatic Huntington's Disease Mice.” Proc Natl Acad Sci USA. 97(12):6809-14.
Bilan et al. 2013. “HyPer-3: a genetically encoded H(2)O(2) probe with improved performance for ratiometric and fluorescence lifetime imaging.” ACS Chem Biol. 8(3):535-42.
Bilan et al. 2014. “Genetically encoded fluorescent indicator for imaging NAD(+)/NADH ratio changes in different cellular compartments.” Biochim Biophys Acta. 1840(3):951-7
Brette et al. 2017. “A Novel Cardiotoxic Mechanism for a Pervasive Global Pollutant.” Sci Rep 7 (January): 41476.
Carlyle et al. 2014. “cAMP-PKA Phosphorylation of Tau Confers Risk for Degeneration in Aging Association Cortex.” Proc Natl Acad Sci USA 111(13): 5036-41.
Chiu et al. 2015. “Elucidating the Role of the A2A Adenosine Receptor in Neurodegeneration Using Neurons Derived from Huntington's Disease iPSCs.” Hum Mol Genet 24(21): 6066-79.
Cubillos-Ruiz et al. 2017. “Tumorigenic and Immunosuppressive Effects of Endoplasmic Reticulum Stress in Cancer.” Cell 168(4): 692-706.
Dana et al. 2014. “Thy1-GCaMP6 Transgenic Mice for Neuronal Population Imaging in Vivo.” PloS One 9(9): e108697.
Day and Davidson. 2009. “The fluorescent protein palette: tools for cellular imaging.” Chem Soc Rev. 2009 October; 38(10): 2887-2921.
Dittmer et al. 2009. “Genetically encoded sensors to elucidate spatial distribution of cellular zinc.” J Biol Chem. 284(24): 16289-16297.
Foufelle et al. 2016. “Role of Endoplasmic Reticulum Stress in Drug-Induced Toxicity.” Pharmacol Res Perspect 4(1): e00211.
Gress et al. 2015. “Cardiotoxic Electrophysiological Effects of the Herbicide ROUNDUP in Rat and Rabbit Ventricular Myocardium In Vitro.” Cardiovasc Toxicol 15(4):324-35.
Grootjans et al. 2016. “The Unfolded Protein Response in Immunity and Inflammation.” Nat Rev Immunol 16(8): 469-84.
Gu et al. 2006. “A novel analytical method for in vivo phosphate tracking.” FEBS Lett. 580(25):5885-93
Halliday et al. 2014. “Targeting the Unfolded Protein Response in Neurodegeneration: A New Approach to Therapy.” Neuropharmacol 76 Pt A (January):169-74.
Hebb et al. 2004. “Striatal Phosphodiesterase mRNA and Protein Levels Are Reduced in Huntington's Disease Transgenic Mice prior to the Onset of Motor Symptoms.” Neuroscience 123 (4): 967-81.
Hung et al. 2011. “Imaging cytosolic NADH-NAD(+) redox state with a genetically encoded fluorescent biosensor.” Cell Metab. 14(4):545-54.
Iwawaki et al. 2003. “A Transgenic Mouse Model for Monitoring Endoplasmic Reticulum Stress.” Nat Med 10(1): 98-102.
Kaper et al. 2007. “Nanosensor detection of an immunoregulatory tryptophan influx/kynurenine efflux cycle.” PLoS Biol. 5(10):e257
Lager et al. 2006. “Conversion of a putative Agrobacterium sugar-binding protein into a FRET sensor with high selectivity for sucrose.” J Biol Chem 281(41):30875-83).
Lindenburg et al. 2013. “MagFRET: the first genetically encoded fluorescent Mg2+ sensor.” PLoS One. 8(12):e82009.
Marambaud et al. 2009. “Calcium Signaling in Neurodegeneration.” Mol Neurodegener 4 (May): 20.
Moore et al. 2016. “Cilia Have High cAMP Levels That Are Inhibited by Sonic Hedgehog-Regulated Calcium Dynamics.” Proc Nat Acad Sci USA 113(46): 13069-74.
Nikolaev et al. 2006. “Fluorescent Sensors for Rapid Monitoring of Intracellular cGMP.” Nat Methods 3(1): 23-25.
Okumuto et al. 2005. “Detection of glutamate release from neurons by genetically encoded surface-displayed FRET nanosensors.” Proc Natl Acad Sci USA. 2005. 102(24):8740-5.
Peschek et al. 2015. “A Conformational RNA Zipper Promotes Intron Ejection during Non-conventional XBP1 mRNA Splicing.” EMBO Reports 16(12): 1688-98.
Pilkis et al. 1992. “Molecular Physiology of the Regulation of Hepatic Gluconeogenesis and Glycolysis.” Annu Rev Physiol 54: 885-909.
Pluquet et al. 2015. “The Unfolded Protein Response and Cellular Senescence. A Review in the Theme: Cellular Mechanisms of Endoplasmic Reticulum Stress Signaling in Health and Disease,” Am J Physiol Cell Physiol 308(6): C415-25.
Rana et al. 2017. “Utilization of iPSC-derived human neurons for high-throughput drug-induced peripheral neuropathy screening.” Toxicol In Vitro.” 45(Pt 1):111-118
Rivolta et al. 2002. “Retinitis Pigmentosa and Allied Diseases: Numerous Diseases, Genes, and Inheritance Patterns.” Hum Mol Gen 11(10): 1219-27.
Rodriguez et al. 2017. “The Growing and Glowing Toolbox of Fluorescent and Photoactive Proteins. Trends Biochem Sci 42(2):111-29.
Rong et al. 2015. “Cell-Based High-Throughput Luciferase Reporter Gene Assays for Identifying and Profiling Chemical Modulators of Endoplasmic Reticulum Signaling Protein, IRE1.” J Biomol Screen 20(10): 1232-45.
San Martin et al. 2013. “A genetically encoded FRET lactate sensor and its use to detect the Warburg effect in single cancer cells.” PLoS One. 8(2):e57712
Santos et al. 2009. “Mechanisms and Implications of Reactive Oxygen Species Generation during the Unfolded Protein Response: Roles of Endoplasmic Reticulum Oxidoreductases, Mitochondrial Electron Transport, and NADPH Oxidase.” Antioxid Redox Signal 11(10): 2409-27.
Scheper et al. 2015. “The Unfolded Protein Response in Neurodegenerative Diseases: A Neuropathological Perspective.” Acta Neuropathol 130(3):315-31.
Shcherbakova et al. 2016. “Bright monomeric near-infrared fluorescent proteins as tags and biosensors for multiscale imaging.” Nat Commun 7:12405
Shaner et al. 2013. “A Bright Monomeric Green Fluorescent Protein Derived from Branchiostoma Lanceolatum.” Nat Methods 10 (5): 407-9.
Shaner et al. 2005. “A Guide to Choosing Fluorescent Proteins.” Nat Methods 2(12):905-9.
Storace et al. 2016. “Toward Better Genetically Encoded Sensors of Membrane Potential.” Trends Neurosci.” 39(5):277-89.
Sugars et al. 2004. “Decreased cAMP response element-mediated transcription: an early event in exon 1 and full-length cell models of Huntington's disease that contributes to polyglutamine pathogenesis.” J Biol Chem 279(6):4988-99.
Sung et al. 1991. “Functional Heterogeneity of Mutant Rhodopsins Responsible for Autosomal Dominant Retinitis Pigmentosa.” Proc Natl Acad Sci USA 88(19):8840-44.
Szymczak et al. 2004. “Correction of Multi-Gene Deficiency in Vivo Using a Single ‘Self-Cleaving’ 2A Peptide-based Retroviral Vector.” Nat Biotechnol 22(5):589-94.
Talbert et al. 2015. “A Multi-Parameter in Vitro Screen in Human Stem Cell-Derived Cardiomyocytes Identifies Ponatinib-Induced Structural and Functional Cardiac Toxicity.” Toxicol Sci 143(1):147-55.
Tallini et al. 2006. “Imaging Cellular Signals in the Heart in Vivo: Cardiac Expression of the High-Signal Ca²⁺ Indicator GCaMP2.” Proc Natl Acad Sci USA 103(12):4753-58.
Tewson et al. 2016. “New DAG and cAMP Sensors Optimized for Live-Cell Assays in Automated Laboratories.” J Biomol Screen 21(3): 298-305.
Uemura et al. 2013. “UBC9 Regulates the Stability of XBP1, a Key Transcription Factor Controlling the ER Stress Response.” Cell Struct Funct 38 (1): 67-79.
van Schadewijk et al. 2012. “A quantitative method for detection of spliced X-box binding protein-1 (XBP1) mRNA as a measure of endoplasmic reticulum (ER) stress.” Cell Stress Chaperones 17(2):275-279.
Wang et al. 2012. “The Impact of the Unfolded Protein Response on Human Disease.” J Cell Biol 197(7):857-67.
Xu et al. 1999. “A bioluminescence resonance energy transfer (BRET) system: application to interacting circadian clock proteins.” Proc Natl Acad Sci USA 96:151-6.
Yoshida et al. 2001. “XBP1 mRNA Is Induced by ATF6 and Spliced by IRE1 in Response to ER Stress to Produce a Highly Active Transcription Factor.” Cell 107(7): 881-91.
Yoshida et al. 2006. “pXBP1(U) Encoded in XBP1 Pre-mRNA Negatively Regulates Unfolded Protein Response Activator pXBP1(S) in Mammalian ER Stress Response.” J Cell Biol 172 (4): 565-75.
Zhao et al. 2011. “An Expanded Palette of Genetically Encoded Ca²⁺ Indicators.” Science 333 (6051): 1888-91.
JP2004081143A
US20040170622A1
US20050250182A1
US20100221743A1
US20110142799
US20110142799A1
U.S. Pat. No. 9,085,791B2
U.S. Pat. No. 9,547,017B2

What is claimed is:

1. A nucleic acid comprising from 5′ to 3′;
a) a first open reading frame encoding a first reporter protein;
b) a linker sequence comprising an intron that is spliced by inositol-requiring enzyme 1 (IRE1) when transcribed to mRNA; and
c) a second open reading frame encoding a second reporter protein, wherein the first and second open reading frames are out of frame from each other.

2. The nucleic acid of claim 1, wherein the intron comprises SEQ ID NO: 1.

3. The nucleic acid of claim 1, wherein the intron comprises SEQ ID NO: 2.

4. The nucleic acid of claim 1, wherein the intron comprises SEQ ID NO: 3.

5. The nucleic acid of any one of claims 1-4, wherein the first reporter protein is a fluorescent or bioluminescent biosensor, the second reporter protein is a fluorescent or bioluminescent biosensor, or both the first and second reporter proteins are fluorescent or bioluminescent biosensors.

6. The nucleic acid of claim 5, wherein the first reporter protein is a fluorescent or bioluminescent biosensor and the second reporter protein is a fluorescent protein.

7. The nucleic acid of claim 5, wherein the first reporter protein is a fluorescent protein and the second reporter protein is a fluorescent or bioluminescent biosensor.

8. The nucleic acid of any one of claims 1-7, wherein the linker sequence comprises one or more sequence elements selected from the group consisting of an IRES, a 2A peptide, and an alternative translation initiation signal.

9. The nucleic acid of any one of claims 1-7, wherein the linker sequence encodes a peptide that links the translated first and second reporter proteins when the intron is spliced from the linker sequence mRNA.

10. The nucleic acid of claim 9, wherein the first reporter protein and the second reporter protein can act as a donor and acceptor pair for resonance energy transfer, wherein the first reporter protein is the donor and the second reporter protein is the acceptor, or the first reporter protein is the acceptor and the second reporter protein is the donor.

11. The nucleic acid of claim 10, wherein the linker sequence further comprises a sequence encoding a caspase cleavage site.

12. The nucleic acid of any one of claims 5-7, wherein the first or second fluorescent or bioluminescent biosensor detects changes in the cellular level of a molecule selected from the group consisting of calcium, cyclic adenosine monophosphate (cAMP), cyclic guanylate monophosphate (cGMP), diacylglycerol, adenosine triphosphate (ATP), adenosine disphosphate (ADP), glucose, ribose, sucrose, glutamate, hydrogen peroxide, lactate, magnesium, oxidized nicotinamide adenine dinucleotide (NAD⁺), non-oxidized nicotinamide adenine dinucleotide (NADH), phosphate, reactive oxygen species, and zinc.

13. The nucleic acid of any one of claims 5-7, wherein the first or second fluorescent biosensor detects changes in the transmembrane voltage of a cell.

14. The nucleic acid of any one of claims 1-4, wherein the first reporter protein is a fluorescent protein, the second reporter protein is a fluorescent protein, or both the first and second reporter proteins are fluorescent proteins.

15. The nucleic acid of any one of claims 1-14, wherein the first open reading frame and the second open reading frame are operatively linked to the same promoter.

16. A nucleic acid comprising from 5′ to 3′;
a) a first exon of a reporter protein;
b) a linker sequence comprising an intron that is spliced by inositol-requiring enzyme 1 (IRE1); and
c) a second exon of the reporter protein, wherein the second exon is in a different reading frame from the first exon,
wherein splicing of the intron in mRNA by IRE1 places the second exon in the same reading frame as the first exon.

17. The nucleic acid of claim 16, wherein the intron comprises SEQ ID NO: 1.

18. The nucleic acid of claim 16, wherein the intron comprises SEQ ID NO: 2.

19. The nucleic acid of claim 16, wherein the intron comprises SEQ ID NO: 3.

20. The nucleic acid of claim 17, wherein the reporter protein is selected from the group consisting of a fluorescent biosensor, a bioluminescent biosensor, and a fluorescent protein.

21. The nucleic acid of any one of claims 16-20, wherein the reporter protein is a fluorescent or bioluminescent biosensor that detects changes in the cellular level of a molecule selected from the group consisting of calcium, cyclic adenosine monophosphate (cAMP), cyclic guanylate monophosphate (cGMP), diacylglycerol, adenosine triphosphate (ATP), adenosine disphosphate (ADP), glucose, ribose, sucrose, glutamate, hydrogen peroxide, lactate, magnesium, oxidized nicotinamide adenine dinucleotide (NAD⁺), non-oxidized nicotinamide adenine dinucleotide (NADH), phosphate, reactive oxygen species, and zinc.

22. The nucleic acid of any one of claims 16-20, wherein the fluorescent or bioluminescent biosensor detects changes in the transmembrane voltage of a cell.

23. A vector comprising the nucleic acid molecule of any one of claims 1-22.

24. A cell comprising the nucleic acid of any one of claims 1-22 or the vector of claim 23.

25. The cell of claim 24, wherein the nucleic acid is inserted into the genome of the cell.

26. A kit comprising the nucleic acid of any one of claims 1-22, the vector of claim 23, or the cell of claim 24 or claim 25.

27. A protein encoded by the nucleic acid of any one of claims 1-22.

28. A method for measuring signaling in a cell, the method comprising:
exposing a cell comprising the nucleic acid of any one of claims 1-15 to light having an excitation wavelength of the first reporter protein, and light having an excitation wavelength of the second reporter protein, and measuring the fluorescence from the cell at the emission wavelength of the first reporter protein and at the emission wavelength of the second reporter protein.

29. The method of claim 28, further comprising contacting the cell with a molecule or organism selected from the group consisting of: a small molecule; a protein; a bacterium; a virus; a protozoan; a worm; or a fungus.

30. The method of claim 28, further comprising exposing the cell to an environmental stressor selected from the group consisting of: a temperature change; a pH change, an osmolarity change; a pressure change; a gravitational force change, and mechanical damage to the cell.

31. A method for measuring signaling in a cell, the method comprising:
exposing a cell comprising the nucleic acid of any one of claims 16-22 to light having an excitation wavelength of the reporter protein, and measuring the fluorescence from the cell at the emission wavelength of the reporter protein.

32. The method of claim 31, further comprising contacting the cell with a molecule or organism selected from the group consisting of: a small molecule; a protein; a bacterium; a virus; a protozoan; a worm; or a fungus.

33. The method of claim 31, further comprising exposing the cell to an environmental stressor selected from the group consisting of: a temperature change; a pH change, an osmolarity change; a pressure change; a gravitational force change, and mechanical damage to the cell.
